(19) United States
(12) Patent Application Publication
(10) Pub. No.: US 2006/0121519 A1
(43) Pub. Date: Jun. 8, 2006

(54) Compositions and methods for purifying and crystallizing molecules of interest

(75) Inventor: Guy Patchornik, Kiryat-Ono (IL)

Correspondence Address: Martin D. Moynihan;PRTSI, Inc., P.O. Box 16446, Arlington, VA, 22215, US

(73) Assignee: Affisink Biotechnology Ltd., Kiryat-Ono (IL)

(21) Appl. No.: 11/330,112

(22) Filed: Jan. 12, 2006

Related U.S. Application Data
(63) Continuation-in-part of application No. PCT/IL04/00669, filed on Mar. 22, 2004.

(30) Foreign Application Priority Data
Jul. 24, 2003 (IL) 157086

(51) Int. Cl. C12Q 1/68; G01N 33/53; G06F 19/00

(52) U.S. Cl. 435/6; 435/7.5; 702/19

(57) ABSTRACT

A composition of matter is provided. The composition includes at least one ligand capable of binding a target molecule or cell of interest, the at least one ligand being attached to at least one coordinating moiety selected capable of directing the composition of matter to form a non-covalent complex when co-incubated with a coordinator ion or molecule. Also provided are methods of using such compositions for target purification, crystallization and immunization.

### RELATED APPLICATIONS

This is a Continuation-In-Part (CIP) of PCT Application No. PCT/IL2004/000669, filed on Jul. 22, 2004, which claims priority from Israel Patent Application No. 157086, filed on Jul. 24, 2003. The contents of the above applications are incorporated herein by reference.

### FIELD AND BACKGROUND OF THE INVENTION

The present invention relates to compositions, which can be used for purifying and crystallizing molecules of interest.

Proteins and other macromolecules are increasingly used in research, diagnostics and therapeutics. Proteins are typically produced by recombinant techniques on a large scale with purification constituting the major cost (up to 60% of the total cost) of the production processes. Thus, large-scale use of recombinant protein products is hindered because of the high cost associated with purification.

Current protein purification methods are dependent on the use of a combination of various chromatography techniques. These techniques separate mixtures of proteins on the basis of their charge, degree of hydrophobicity or size among other characteristics. Several different chromatography resins are available for use with each of these techniques, allowing accurate tailoring of the purification scheme to the particular protein targeted for isolation. The essence of each of these separation methods is that proteins can be caused either to move at different rates down a long column, achieving a physical separation that increases as they pass further down the column, or to adhere selectively to the separation medium, enabling differential elution by different solvents. In some cases, the column is designed such that impurities bind thereto while the desired protein is found in the “flow-through.”

Affinity precipitation (AP) is the most effective and advanced approach for protein precipitation [Mattiasson (1998); Hilbrig and Freitag (2003) J Chromatogr B Analyt Technol Biomed Life Sci. 790(1-2):79-90]. Current state of the art AP employs ligand coupled “smart polymers”. “Smart polymers” [or stimuli-responsive “intelligent” polymers or Affinity Macro Ligands (AML)] are polymers that respond with large property changes to small physical or chemical stimuli, such as changes in pH, temperature, radiation and the like. These polymers can take many forms; they may be dissolved in an aqueous solution, adsorbed or grafted on aqueous-solid interfaces, or cross-linked to form hydrogels [Hoffman J Controlled Release (1987) 6:297-305; Hoffman Intelligent polymers. In: Park K, ed. Controlled drug delivery. Washington: ACS Publications, (1997) 485-98; Hoffman Intelligent polymers in medicine and biotechnology. Artif Organs (1995) 19:458-467]. Typically, when the polymer's critical response is stimulated, the smart polymer in solution will show a sudden onset of turbidity as it phase-separates; the surface-adsorbed or grafted smart polymer will collapse, converting the interface from hydrophilic to hydrophobic; and the smart polymer (cross-linked in the form of a hydrogel) will exhibit a sharp collapse and release much of its swelling solution. These phenomena are reversed when the stimulus is reversed, although the rate of reversion often is slower when the polymer has to redissolve or the gel has to re-swell in aqueous medium.

“Smart” polymers may be physically mixed with, or chemically conjugated to, biomolecules to yield a large family of polymer-biomolecule systems that can respond to biological as well as to physical and chemical stimuli. Biomolecules that may be polymer-conjugated include proteins and oligopeptides, sugars and polysaccharides, single- and double-stranded oligonucleotides and DNA plasmids, simple lipids and phospholipids, and a wide spectrum of recognition ligands and synthetic drug molecules.

A number of structural parameters control the ability of smart polymers to specifically precipitate proteins of interest; smart polymers should contain reactive groups for ligand coupling; not interact strongly with the impurities; make the ligand available for interaction with the target protein; give complete phase separation of the polymer upon a change of medium property; form compact precipitates; exclude trapping of impurities into the gel structure and be easily solubilized after the precipitate is formed.

Although many different natural as well as synthetic polymers have been utilized in AP [Mattiasson (1998) J. Mol. Recognit. 11:211] the ideal smart polymers remain elusive, as affinity precipitations performed with currently available smart polymers, fail to meet one or several of the above-described requirements [Hlibrig and Freitag (2003), supra].

The availability of efficient and simple protein purification techniques may also be useful in protein crystallization, in which protein purity extensively affects crystal growth. The conformational structure of proteins is a key to understanding their biological functions and to ultimately designing new drug therapies. The conformational structures of proteins are conventionally determined by x-ray diffraction from their crystals. Unfortunately, growing protein crystals of sufficient high quality is very difficult in most cases, and such difficulty is the main limiting factor in the scientific determination and identification of the structures of protein samples. Prior art methods for growing protein crystals from super-saturated solutions are tedious and time-consuming, and less than two percent of the over 100,000 different proteins have been grown as crystals suitable for x-ray diffraction studies.

Membrane proteins present the most challenging group of proteins for crystallization. The number of 3D structures available for membrane proteins is still around 20 while the number of membrane proteins is expected to constitute a third of the proteome. Numerous obstacles need to be traversed when wishing to crystallize a membrane protein. These include, low abundance of proteins from natural sources, the need to solubilize hydrophobic membrane proteins from their native environment (i.e., the lipid bilayer) and their tendency to denaturate, aggregate and/or degrade in the detergent solution. The choice of the solubilizing detergent presents another problem as some detergents may interfere with binding of a stabilizing partner to the target protein.

Two approaches have been attempted in the crystallization of membrane proteins.

Until very recently, the majority of X-ray crystal structures of membrane proteins have been determined using crystals grown directly from solutions of protein-detergent complexes. Crystal growth of protein-detergent complexes can be considered equivalent to that of soluble proteins only the solute being crystallized is a complex of protein and detergent, rather than solely protein. The actual lattice contacts are formed by protein-protein interactions, although crystal packing brings the detergent moieties into close apposition as well. In order to increase the surface area available to make these protein-protein contacts studies suggested adding an antibody fragment which will increase the chances of producing crystals [Hunte and Michel (2002) Curr. Opin. Struct. Biol. 12:503-508]. However, applying this technology to various membrane proteins is difficult as it requires the generation of monoclonal antibodies, which are specific to each membrane protein.

Furthermore, it is argued that no detergent micelle can fully and accurately reproduce the lipid bilayer environment of the protein.

Thus, efforts to crystallize membrane proteins must be directed towards producing crystals within a bilayer environment. A number of attempts have been made to generate crystals of membrane proteins using this approach. These include the generation of crystals of bacteriorhodopsin grown in the presence of a lipidic cubic phase, which forms gel-like substance containing continuous bilayer structures [Landau and Rosenbuch (1996) Proc. Natl. Acad. Sci. USA 93:14532-14535] and crystallization in cubo which was proven successful in the crystallization of archaeal seven-transmembrane helix proteins [Gordeliy (2002) Nature 419:484-487; Luecke (2001) Science 293:1499-1503; Kolbe (2000) Science 288:1390-1396; Royant (2001) Proc. Natl. Acad. Sci. USA 98:10131-10136]. However, crystals of other membrane proteins using the in cubo approach were not of as high a quality as crystals grown directly from protein-detergent complex solutions [Chiu (2000) Acta. Crystallogr. D. 56:781-784].

There is thus a widely recognized need for, and it would be highly advantageous to have, compositions and methods using same for the purification and crystallization of molecules which are devoid of the above limitations.

### SUMMARY OF THE INVENTION

According to one aspect of the present invention there is provided a composition of matter comprising at least one ligand capable of binding a target molecule or cell of interest, the at least one ligand being attached to at least one coordinating moiety selected capable of directing the composition of matter to form a non-covalent complex when co-incubated with a coordinator ion or molecule.

According to another aspect of the present invention there is provided a method of purifying a target molecule or cell of interest, the method comprising: (a) contacting a sample including the target molecule or cell of interest with a composition including: (i) at least one ligand capable of binding the target molecule or cell of interest, the at least one ligand being attached to at least one coordinating moiety; and (ii) a coordinator capable of non-covalently binding the at least one coordinating moiety, the at least one coordinating moiety and the coordinator being capable of forming a complex when co-incubated; and (b) collecting a precipitate including the complex bound to the target molecule or cell of interest, thereby purifying the target molecule or cell of interest.

According to further features in preferred embodiments of the invention described below, the method further comprising recovering the molecule of interest from the precipitate.

According to yet another aspect of the present invention there is provided a method of detecting predisposition to, or presence of a disease associated with a molecule of interest in a subject, the method comprising contacting a biological sample obtained from the subject with a composition including: (i) at least one ligand capable of binding the molecule of interest, the at least one ligand being attached to at least one coordinating moiety; and (ii) a coordinator capable of non-covalently binding the at least one coordinating moiety, the at least one coordinating moiety and the coordinator being capable of forming a complex when co-incubated, wherein formation of the complex including the molecule of interest is indicative of predisposition to, or presence of the disease associated with the molecule of interest in the subject.

According to still another aspect of the present invention there is provided a composition for crystallizing a molecule of interest, the composition comprising: (i) at least one ligand capable of binding the molecule of interest, the at least one ligand being attached to at least one coordinating moiety; and (ii) a coordinator capable of non-covalently binding the at least one coordinating moiety, wherein the at least one coordinating moiety and the coordinator are capable of forming a complex when co-incubated and whereas the composition is selected so as to define the relative spatial positioning and orientation of the molecule of interest when bound thereto, thereby facilitating formation of a crystal therefrom under inducing crystallization conditions.

According to an additional aspect of the present invention there is provided a method of crystallizing a molecule of interest, the method comprising contacting a sample including the molecule of interest with a crystallizing composition including: (i) at least one ligand capable of binding the molecule of interest, the at least one ligand being attached to at least one coordinating moiety; and (ii) a coordinator capable of non-covalently binding the at least one coordinating moiety, wherein the at least one coordinating moiety and the coordinator are capable of forming a complex when co-incubated and whereas the crystallizing composition is selected so as to define the relative spatial positioning and orientation of the molecule of interest when bound thereto, thereby facilitating formation of a crystal therefrom under inducing crystallization conditions.

According to yet an additional aspect of the present invention there is provided a composition-of-matter comprising a molecule having a first region capable of binding a molecule of interest and a second region capable of binding a coordinator ion or molecule, the second region being designed such that the molecule forms a polymer when exposed to the coordinator ion or molecule.

According to still an additional aspect of the present invention there is provided a method of depleting a target molecule or cell of interest from a sample, the method comprising: (a) contacting the sample including the target molecule or cell of interest with a composition including: (i) at least one ligand capable of binding the molecule of interest, the at least one ligand being attached to at least one coordinating moiety; and (ii) a coordinator capable of non-covalently binding the at least one coordinating moiety, the at least one coordinating moiety and the coordinator being capable of forming a complex when co-incubated; and (b) removing a precipitate including the complex bound to the target molecule or cell of interest to thereby deplete the target molecule or cell of interest from the sample.

According to a further aspect of the present invention there is provided a method of enhancing immunogenicity of a target molecule of interest, the method comprising contacting the target molecule of interest with a composition including: (i) at least one ligand capable of binding the target molecule of interest, the at least one ligand being attached to at least one coordinating moiety; and (ii) a coordinator capable of non-covalently binding the at least one coordinating moiety, wherein contacting is effected such that the at least one coordinating moiety and the coordinator forms a complex including the target molecule of interest, thereby enhancing immunogenicity of the target molecule of interest.

According to still further features in the described preferred embodiments the molecule of interest is selected from the group consisting of a protein, a nucleic acid sequence, a small molecule chemical and an ion.

According to still further features in the described preferred embodiments the at least one ligand is selected from the group consisting of a growth factor, a hormone, a nucleic acid sequence, an antibody, an epitope tag, an avidin, a biotin, a enzymatic substrate and an enzyme.

According to still further features in the described preferred embodiments the at least one ligand is attached to the at least one coordinating moiety via a linker.

According to still further features in the described preferred embodiments the coordinating moiety is selected from the group consisting of a chelator, a biotin, a nucleic acid sequence, an epitope tag, an electron poor molecule and an electron-rich molecule.

According to still further features in the described preferred embodiments the coordinator ion or molecule is selected from the group consisting of a metal ion, an avidin, a nucleic acid sequence, an electron poor molecule and an electron-rich molecule.

The present invention successfully addresses the shortcomings of the presently known configurations by providing compositions and methods for the purification of molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-f schematically illustrate several configurations of the compositions of the present invention. FIGS. 1a-c show ligands bound to two coordinating moieties. FIGS. 1d-f show ligands bound to multiple coordinating moieties. Z denotes the coordinating moiety;

FIGS. 2a-b schematically illustrate precipitation of a target molecule using the compositions of the present invention. A ligand covalently attached to a bis-chelator is incubated in the presence of a target molecule (FIG. 2a). Addition of a metal (M⁺, M²⁺, M³⁺, M⁴⁺) binds the chelator and forms a matrix including the target molecule non-covalently bound to the metal ion (FIG. 2b);

FIGS. 3a-e schematically illustrate stepwise recovery of the target molecule from the precipitate. FIG. 3a shows the addition of a free chelator, which competes with the binding of the ligand-bound chelator to the metal. FIG. 3b shows gravity-based separation of the ligand-bound target molecule from the free competing chelator and the complexed metal (FIG. 3c). FIG. 3d shows loading of the ligand-bound target molecule on an immobilized metal column to allow binding of the complex. Under proper elution conditions the target molecule is eluted while the ligand-coordinating moiety molecule is not. A desalting stage may be added for further purification of the target molecule. Regeneration of the ligand-chelator molecule is achieved by addition of a competing chelator to the column, followed by dialysis or ultrafiltration (FIG. 3e);

FIG. 4 schematically illustrates direct elution of the target molecule from the precipitate, wherein the chelator-metal complex is maintained, while binding between the target molecule and the ligand decreases;

FIG. 5 schematically illustrates regeneration of the precipitating unit (i.e., ligand-coordinating moiety) following elution of the target molecule. In this case, recovery is achieved by the addition of a competing chelator and application of an appropriate separation procedure, such as, dialysis and ultrafiltration;

FIGS. 6a-c schematically illustrate precipitation of a target molecule using nucleic acid sequences as the coordinating moiety. A ligand with a covalently bound bis-nucleotide sequence (coordinating moiety) is incubated in the presence of a target molecule (FIG. 6a). Addition of a complementary sequence results in the formation of matrix including ligand-coordinating moiety:target molecule:the complementary sequence (coordinator molecule, FIG. 6b). Non-symmetrical coordinating sequences are shown as well (FIG. 6c);

FIGS. 7a-b schematically illustrate precipitation of a target molecule using biotin as the coordinating moiety. A ligand with a covalently bound bis-biotin or biotin derivative such as: DSB-X Biotin is incubated in the presence of a target molecule (FIG. 7a). Introduction of avidin (or its derivatives) creates a network comprising ligand-coordinating moiety (biotin):target molecule:avidin (FIG. 7b);

FIGS. 8a-c schematically illustrate precipitation of a target molecule using electron rich molecules as the coordinating moiety. A ligand with a covalently bound bis-electron rich entity is incubated in the presence of a target molecule (FIG. 8a). Addition of a bis (also tris, tetra) electron poor derivative with the propensity to form a complex results in a non-covalent network comprising ligand-coordinating moiety (electron poor molecule):target molecule:bis-electron poor moiety (FIG. 8b). The picric acid and indole system can also be used according to the present invention (FIG. 8c);

FIG. 9 schematically illustrates precipitation of a target antibody with protein A (ProA) bound used as a ligand. Addition of an appropriate coordinator results in a network of: Protein A-coordinating moiety:coordinator:target molecule;

FIGS. 10a-b schematically illustrate the use of the complexes of the present invention for crystallization of membrane proteins. The general formation of 2D (or 3D) structures in the presence of crystallizing composition is presented, where the coordinators are not interconnected between themselves (FIG. 10a). A more detailed example utilizing a specific ligand modified with two antigens, and a monoclonal antibody (mAb) directed at the specific antigen, serving as the coordinator, is illustrated in FIG. 10b;

FIGS. 11a-b schematically illustrate the use of metallo complexes (FIG. 11a) and nucleo-complexes (FIG. 11b) for the formation of crystals of membrane proteins;

FIG. 11c schematically illustrates a three-dimensional membrane complex using the compositions of the present invention. The hydrophobic domain of the protein is surrounded by detergent micelles. Z denotes a multi valent coordinator (i.e., at least bi-valent coordinator);

FIG. 12 schematically illustrates the formation of a non-covalent composition consisting of three ligands bound to a single metal coordinator, through suitable chelators which are bound to the ligands through covalent linkers;

FIGS. 13a-b schematically illustrate the modification of three ligands of interest to include the hydroxamate derivatives (FIG. 13a), such that a tri-non-covalent ligand complex is formed in the presence of Fe³⁺ ions (FIG. 13b);

FIG. 14 schematically illustrates a two-step synthesis procedure for the generation of ligand-chelator molecules;

FIGS. 15a-b schematically illustrate the formation of di (FIG. 15a) and tri (FIG. 15b) non-covalent ligands, by utilizing the same ligand-linker-chelator molecule, while changing only, the cation present in the medium;

FIGS. 16a-c schematically illustrate the compositions of the present invention coordinated by electron poor/rich relations. By modifying a ligand with an electron poor moiety (FIG. 16a) and synthesizing a tri covalent electron rich moiety (FIG. 16b), a complex of the structure seen in FIG. 16c is formed;

FIG. 17 schematically illustrates a two step synthesis process for the preparation of ligand-electron rich or ligand-electron poor derivatives;

FIG. 18 schematically illustrates the use of peptides for the formation of ligand complexes utilizing electron rich and electron poor moieties;

FIG. 19 schematically illustrates the formation of ligand complexes which utilize a chelator-metal as well as electron rich and poor relationships;

FIG. 20 schematically illustrates a single step synthesis procedure for the preparation of a chelator-electron poor derivative;

FIGS. 21a-b schematically illustrate formation of di and tri non-covalent electron poor moieties by utilizing the same chelator-electron poor (catechol-TNB) derivative and changing only the cation in the medium;

FIGS. 22a-b schematically illustrate the addition of a peptide containing an electron rich moiety to form a dimer and a trimer;

FIGS. 23a-b schematically illustrate the formation of a polymer complex by the addition of a composition including ligand attached to two chelators which are coordinated through electron rich/poor relations;

FIG. 24 schematically illustrates one possibility of limiting the freedom of motion of non-covalent protein dimers. After non-covalent dimmers are formed via a ligand-linker-chelator with the addition of an appropriate metal, the addition of a covalent electron poor moiety [e.g. trinitrobenzene-trinitrobenzene (TNB-TNB)] leads to the simultaneous binding of two accessible electron rich residues (e.g. Trp) on two adjacent proteins thereby imposing motion constraints and allowing formation of a crystal structure;

FIG. 25 schematically illustrates chelators and metals, which can be used as the coordinating moiety and coordinator ion, respectively, in the compositions of the present invention;

FIG. 26 schematically illustrates electron rich and electron poor moieties which can be used as the coordinating moiety in the compositions of the present invention;

FIGS. 27a-b illustrate purification of rabbit IgG from normal rat kidney (NRK) cell lysate (FIG. 27a) or from mouse myoblasts (C2) cell lysate (FIG. 27b), utilizing Desthiobiotinylated protein A (DB-ProA) and free avidin. FIG. 27a-lane 1 rabbit IgG; lane 2 DB-ProA; lane 3 NRK cell lysate; lane 4 mixture of rabbit IgG, DB-ProA and NRK cell lysate; lane 5 recovered IgG (yield: ˜90% by densitometry); lane 6 content of supernatant after specific precipitation of the IgG from the cell lysate.

FIG. 27b-lane 1 rabbit IgG; lane 2 DB-ProA; lane 3 C2 cell lysate; lane 4 mixture of rabbit IgG, DB-ProA and C2 cell lysate; lane 5 recovered IgG (yield: ˜90% by densitometry); lane 6 content of supernatant after specific precipitation of the IgG from the cell lysate;

FIG. 28 illustrates purification of rabbit IgG from E. coli cell lysate, utilizing desthiobiotinylated protein A (DB-ProA) and free avidin. Lane 1 rabbit IgG; lane 2 DB-ProA; lane 3 E. coli cell lysate; lane 4 mixture of rabbit IgG, DB-Pro A and E. coli cell lysate; lane 5 Biorad prestained protein markers; lane 6 recovered IgG (yield: 85% by densitometry); lane 7 content of supernatant after specific precipitation of the IgG from the cell lysate;

FIG. 29a illustrates the effect of increase background contamination (BSA) on the precipitation process. Lane 1 rabbit IgG; lanes 2-5 constant concentration of rabbit IgG and DB-ProA in the presence of increase BSA concentration; Lane 6 Biorad prestained protein standards; lanes 2P-5P recovered IgG from pellets generated in lanes 2-5 respectively (yield: 80-85% by densitometry);

FIG. 29b illustrates the effect of increase background contamination (E. coli lysate) on the precipitation process. Lane 1 rabbit IgG; lane 2 DB-ProA; lanes 3-5 constant concentration of rabbit IgG and DB-ProA in the presence of increased E. coli cell lysate concentrations; lanes 3P-5P recovered IgG from pellets generated in lanes 3-5, respectively (yield: 80-85% by densitometry);

FIG. 30a illustrates purification of rabbit IgG from E. coli cell lysate utilizing Protein A modified with the strong chelator catechol (ProA-CAT) and Fe³⁺ ions. Lane 1 rabbit IgG; lane 2 native Protein A; lane 3 ProA-CAT; lane 4 E. coli cell lysate; lane 5 rabbit IgG, ProA-CAT and E. coli cell lysate; lane 6 recovered rabbit IgG; lane 7 content of supernatant after addition of Fe³⁺ ions to the mixture in lane 5;

FIG. 30b illustrates the effect of increased background contamination on the precipitation process. Lane 1 rabbit IgG; lane 2 ProA-CAT; lanes 3-5 constant concentration of rabbit IgG and ProA-CAT in the presence of increased E. coli lysate concentrations; lanes 3P-5P recovered IgG from pellets generated in lanes 3-5, respectively;

FIGS. 31a-d illustrate antibody purification utilizing a modified Protein A (ProA-CAT) and Fe³⁺ ions. FIG. 31a—specific binding of ProA-CAT to the target IgG leads to the formation of the: [ProA-CAT:target IgG] soluble complex. FIG. 31b-addition of Fe³⁺ ions to the complex shown in FIG. 31a generates insoluble macro-complexes containing the target IgG. Impurities, left in the supernatant are discarded via centrifugation. FIG. 31c—target IgG is eluted under acidic conditions without dissociating the [ProA-CAT:Fe³⁺] macro-complex of the insoluble pellet. FIG. 31d-Regeneration of ProA-CAT in the presence of strong metal chelators which compete for the complexed Fe³⁺ ions thereby dissociating the macro-complex (i.e., pellet). The complexed Fe³⁺ ions and free chelators are excluded by dialysis while the free ProA-CAT can be reused;

FIGS. 32a-c illustrate a comparison of the basic chemical architecture of affinity chromatography (AC), affinity precipitation (AP) and affinity sinking (AS). FIG. 32a—Ligands in AC are immobilized to non-soluble polymeric matrixes. FIG. 32b—Ligands in AP are immobilized to water soluble polymers which would change reversibly to water in-soluble upon a physiochemical change such as low pH. FIG. 32c—Ligands in AS are not immobilized but modified with a complexing entity enabling their precipitation upon addition of an appropriate Mediator. Thus, no polymeric entity is present within the precipitation process and ligands are free in the medium;

FIGS. 33a-b schematically illustrate positive or negative cell selection (FIG. 33a) and virus depletion (FIG. 33b), utilizing a core complex comprised of [DB-ProA—avidin];

FIG. 34 illustrates simultaneous depletion of several impurities upon addition of different biotinylated ligands and free avidin. The resulting supernatant in stage C. contains enriched mixture of target proteins whereas impurities are left insoluble in the pellet;

FIG. 35 illustrates purification of fusion proteins with a modified human IgG (hIgG) and an appropriate transition metal;

FIG. 36 illustrates covalent modification of a protein (e.g. Ovalbumin) with a small ligand (e.g. peptide) and a complexing entity (e.g. desthiobiotin) would lead to a modified protein (b) possessing multi-complexing features. Its incubation in a medium containing a Target would lead to specific binding of the Target (c) and precipitation of the latter complex upon addition of free Avidin (d). Thus, the Target is specifically precipitated whereas impurities are left soluble in the supernatant and are excluded. Elution of the Target is obtained by incubating the above macro-complex under conditions favoring dissociation of the [Ovalbumin-Ligand:Target] complex while maintaining the: [Ovalbumin-Desthiobiotin:avidin] complex, intact;

FIG. 37 illustrates purification of an Anti-FITC mAb utilizing modified ovalbumin and free avidin. Lane 1—native ovalbumin; lane 2—modified ovalbumin; lane 3—mAb Anti-FITC; lane 4—mixture the mAb and the modified ovalbumin; lane 5—content of supernatant after addition of avidin to lane 4 in the absence of free Fluorescein; lane 6—content of supernatant after addition of avidin to lane 4 in the presence of Fluorescein; lane 7—recovered mAb from the pellet generated in the absence of free Fluorescein; lane 8—recovered mAb from the pellet generated in the presence of free Fluorescein;

FIG. 38 illustrates Purification of His-Tag-Target utilizing non-immobilized Ovalbumin-NTA-Desthiobioitin multi-ligand. Modification of a protein (e.g. Ovalbumin) with a metal chelator (e.g. NTA) and desthiobiotin generates the non-immobilized modified ligand (b). Incubation of the above under proper conditions (e.g. low imidazole concentration); an appropriate metal (e.g. Ni2+, Co2+) and a medium containing the His-Tag-Target will lead to specific binding (c). Addition of free avidin will generate insoluble macro-complexes that will precipitate together with the His-Tag-Target (d). Elution of the His-Tag-Target could then be performed leaving the: [modified ovalbumin:avidin] macro-complex in the pellet; and

FIG. 39 illustrates gel chromatography of a precipitate obtained from a regular network and defective network.

### DESCRIPTION OF THE PREFERRED EMBODIMENTS

The present invention is of compositions, which can be used for purifying and crystallizing molecules of interest.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cost effective commercial-scale production of proteins, such as therapeutic proteins, depends largely on the development of fast and efficient methods of purification since it is the purification step which typically contributes most of the cost involved in large scale production of proteins.

There is thus, a need for simple, cost effective processes, which can be used to purify proteins and other commercially important molecules.

The state of the art approach in protein purification is Affinity Precipitation (AP) which is based on the use of “smart” polymers coupled to a recognition unit, which binds the protein of interest. These smart polymers respond to small changes in environmental stimuli with large, sometimes discontinuous changes in their physical state or properties, resulting in phase separation from aqueous solution or order-of-magnitude changes in hydrogel size and precipitation of the molecule of interest. However, at present, the promise of smart polymers has not been realized due to several drawbacks including, entrapment of impurities during the precipitation process, adsorption of impurities to the polymeric matrix, decreased affinity of the protein recognition unit and working conditions which may lead to a purified protein with reduced activity.

While reducing the present invention to practice, the present inventors designed novel compositions, which can be used for cost-effective and efficient purification of proteins as well as other molecules and cells of interest.

As is illustrated hereinbelow and in the Examples section which follows, the compositions of the present invention specifically bind target molecules to form non-covalent complexes which can be precipitated and collected under mild conditions. Furthermore, contrary to prior art purifying compositions, the compositions of the present invention are not immobilized (such as to a smart polymer) which reduces affinity of the ligand towards the target molecule, limits the amount of ligand used, necessitates the use of sophisticated laboratory equipment (HPLC) requiring high maintenance, leads to column fouling and limits column usage to a single covalently bound ligand.

Thus, according to one aspect of the present invention there is provided a composition-of-matter, which is suitable for purification of a target molecule or cell of interest.

The target molecule can be a macromolecule such as a protein, a carbohydrate, a glycoprotein or a nucleic acid sequence (e.g. DNA such as plasmids, RNA) or a small molecule such as a chemical. Although most of the examples provided herein describe proteinacious target molecules, it will be appreciated that the present invention is not limited to such targets.

The target cell can be a eukaryotic cell, a prokaryotic cell or a viral cell.

The composition-of-matter of the present invention includes at least one ligand capable of binding the molecule or cell of interest and at least one coordinating moiety which is selected capable of directing the composition of matter to form a non-covalent complex when co-incubated with a coordinator ion or molecule.

As used herein the term “ligand” refers to a synthetic or a naturally occurring molecule preferably exhibiting high affinity (e.g. K_{D}<10⁻⁵) binding to the target molecule of interest and as such the two are capable of specifically interacting. When the target of interest is a cell, the ligand is selected capable of binding a protein, a carbohydrate or chemical, which is expressed on the surface of the cell (e.g. cellular marker). Preferably, ligand binding to the molecule or cell of interest is a non-covalent binding. The ligand according to this aspect of the present invention may be mono, bi (antibody, growth factor) or multi-valent ligand and may exhibit affinity to one or more molecules or cells of interest (e.g. bi-specific antibodies). Examples of ligands which may be used in accordance with the present invention include, but are not limited to, antibodies, mimetics (e.g. Affibodies® see: U.S. Pat. Nos. 5,831,012, 6,534,628 and 6,740,734) or fragments thereof, epitope tags, antigens, biotin and derivatives thereof, avidin and derivatives thereof, metal ions, receptors and fragments thereof (e.g. EGF binding domain), enzymes (e.g. proteases) and mutants thereof (e.g. catalytic inactive), substrates (e.g. heparin), lectins (e.g. concanavalin A), carbohydrates (e.g. heparin), nucleic acid sequences [e.g. aptamers and Spiegelmers [Wlotzka® (2002) Proc. Natl. Acad. Sci. USA 99:8898-02], dyes which often interact with the catalytic site of an enzyme mimicking the structure of a natural substrate or co-factor and consisting of a chromophore (e.g. azo dyes, anthraquinone, or phathalocyanine), linked to a reactive group (e.g. a mono- or dichlorotriazine ring, see, Denzili (2001) J Biochem Biophys Methods. 49(1-3):391-416), small molecule chemicals, receptor ligands (e.g. growth factors and hormones), mimetics having the same binding function but distinct chemical structure, or fragments thereof (e.g. EGF domain), ion ligands (e.g. calmodulin), protein A, protein G and protein L or mimetics thereof (e.g. PAM, see Fassina (1996) J. Mol. Recognit. 9:564-9], chemicals (e.g. cibacron Blue which bind enzymes and serum albumin; amino acids e.g. lysine and arginine which bind serine proteases) and magnetic molecules such as high spin organic molecules and polymers (see http://www.chem.unl.edu/rajca/highspin.html).

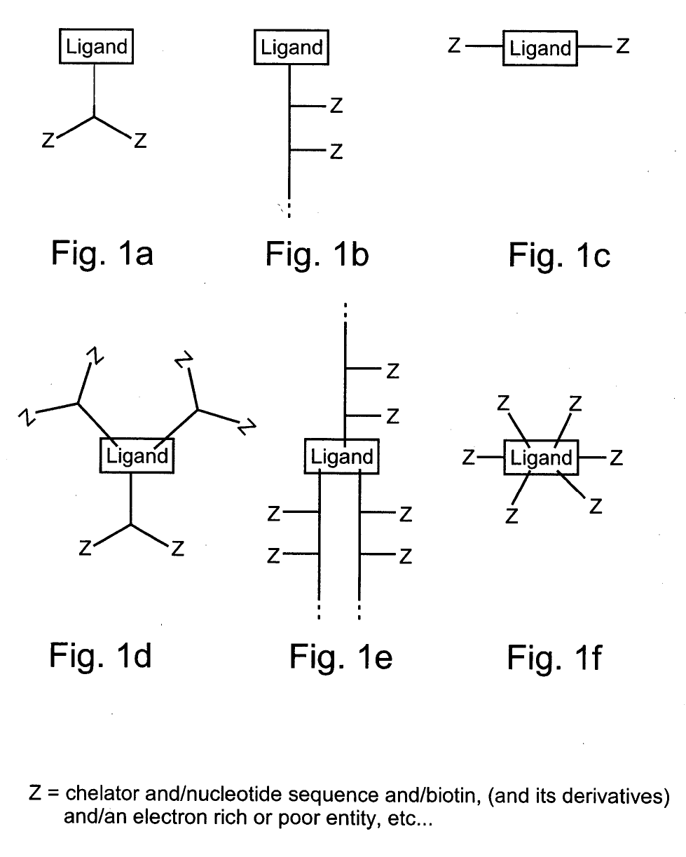

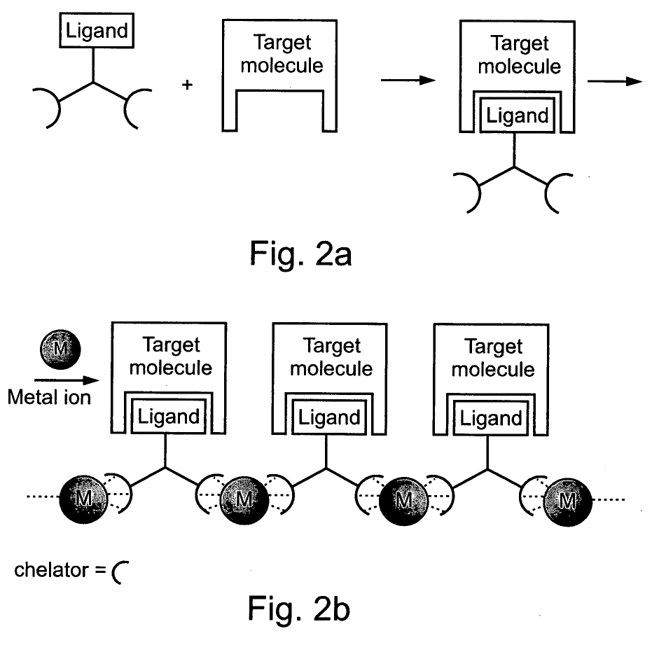

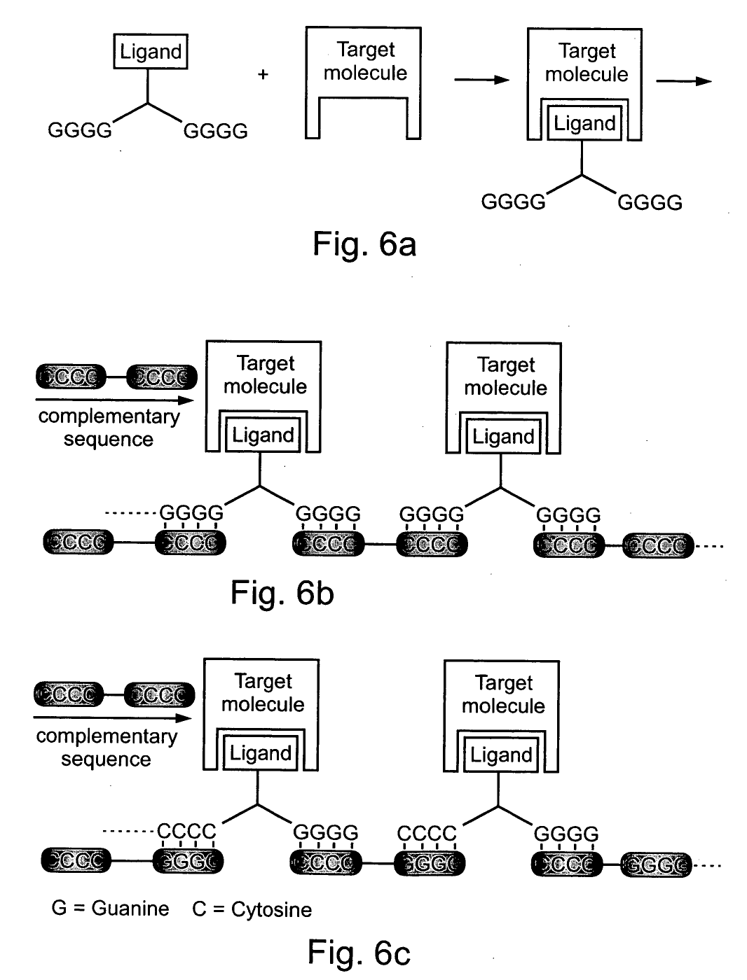

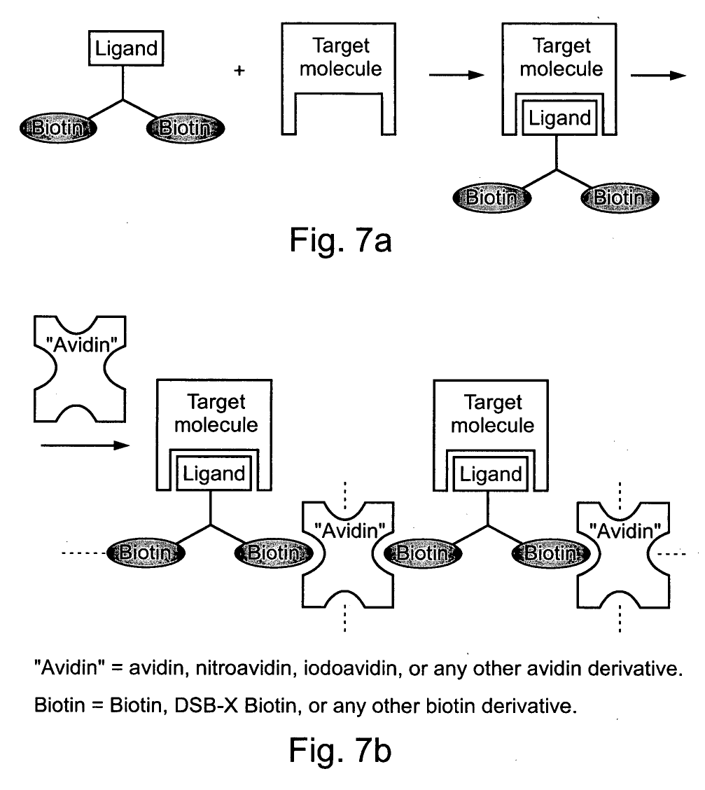

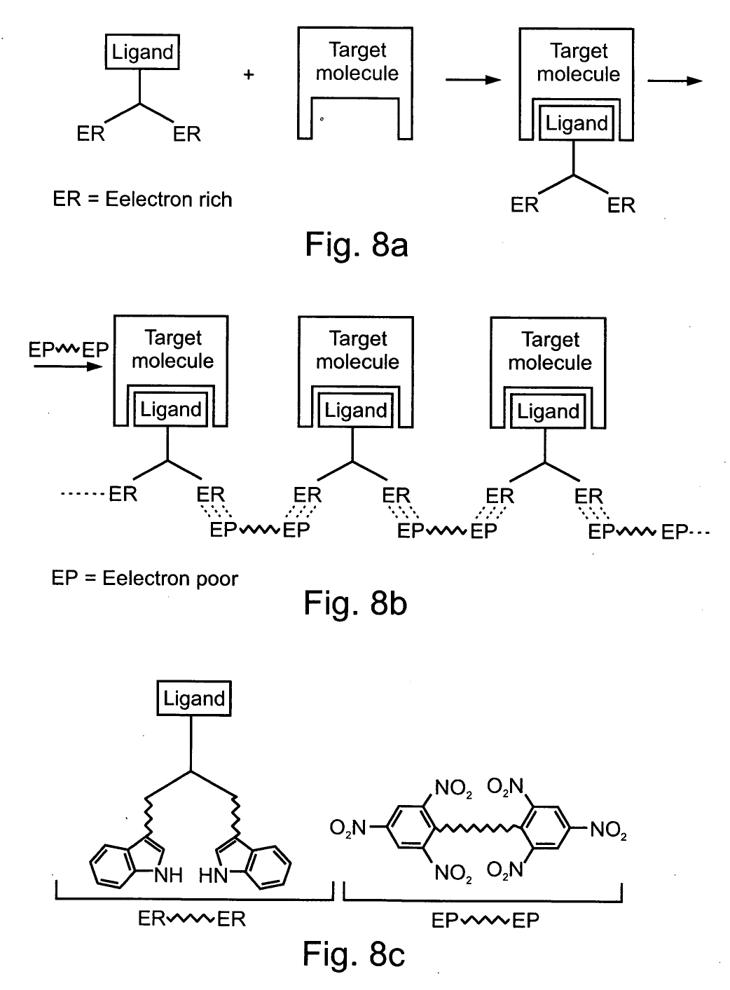

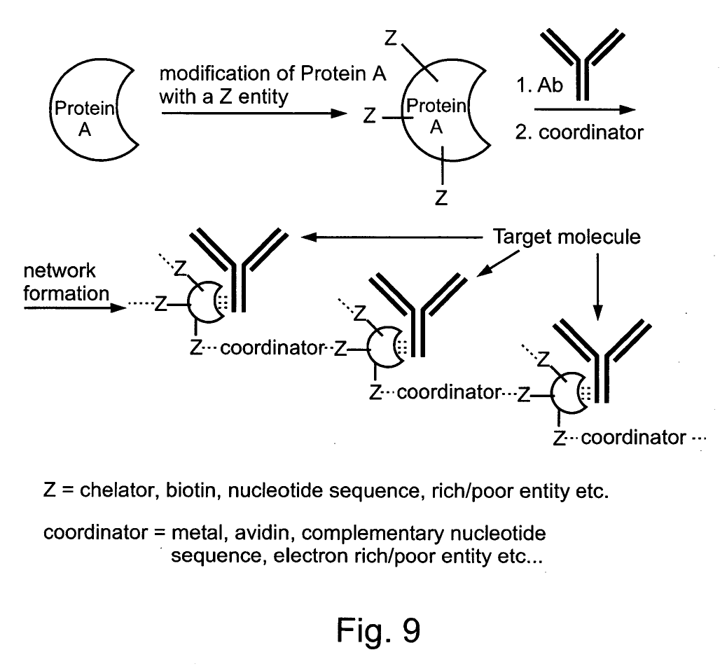

As used herein the phrase “coordinating moiety” refers to any molecule having sufficient affinity (e.g. K_{D}<10⁻⁵) to a coordinator ion or molecule. The coordinating moiety can direct the composition of matter of this aspect of the present invention to form a non-covalent complex when co-incubated with a coordinator ion or molecule. Examples of coordinating moieties which can be used in accordance with the present invention include but are not limited to, epitopes (antigenic determinants antigens to which the paratope of an antibody binds), antibodies, chelators (e.g. His-tag, see other example in Example 1 of the Examples section which follows, FIGS. 1, 25 and 26), biotin (see FIG. 7), nucleic acid sequences (see FIG. 6), protein A or G (FIG. 9), electron poor molecules and electron rich molecules (see Example 2 of the Examples section which follows and FIG. 8) and other molecules described hereinabove (see examples for ligands).

It will be appreciated that a number of coordinating moieties can be bound to the ligand described above (see FIGS. 1a-f).

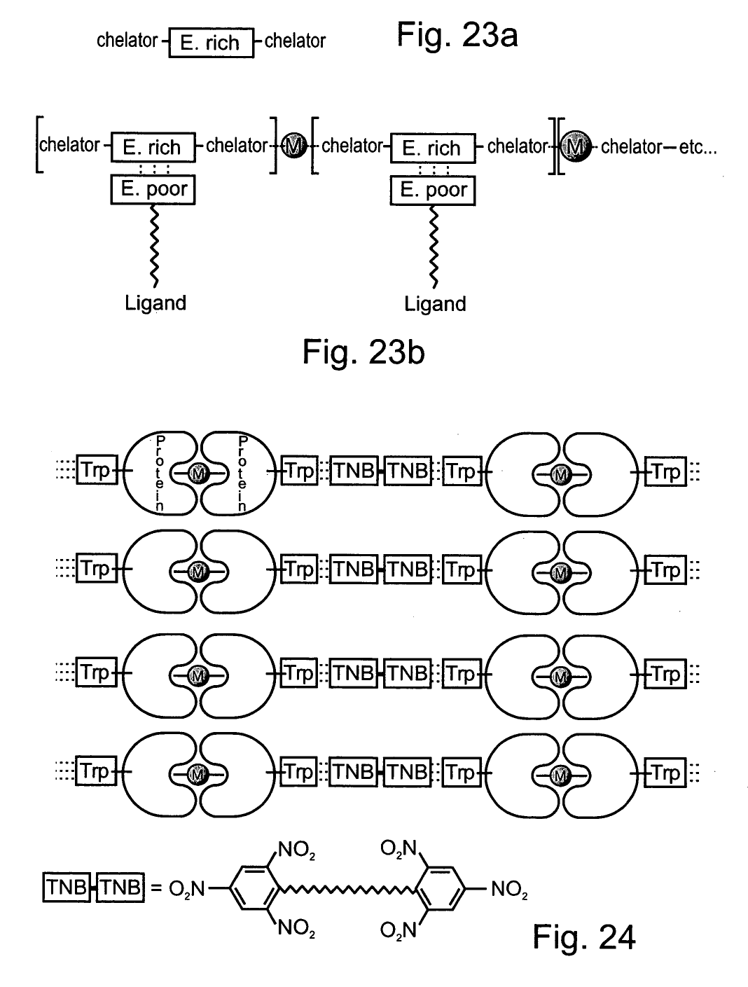

It will be further appreciated that different coordinating moieties can be attached to the ligand such as a chelator and an electron rich/poor molecule to form a complex such as is shown in FIG. 19. Such a combination of binding moieties may mediate the formation of polymers or ordered sheets (i.e., networks) containing the molecule of interest as is illustrated in FIGS. 23a-b and 24, respectively.

To avoid competition and/or further problems in the recovery of the molecule of interest from the complex, the coordinating moiety is selected so as to negate the possibility of coordinating moiety-ligand interaction or coordinating moiety-target molecule interaction. For example, if the ligand is an antigen having an affinity towards an immunoglobulin of interest than the coordinating moiety is preferably not an epitope tag or an antibody capable of binding the antigen.

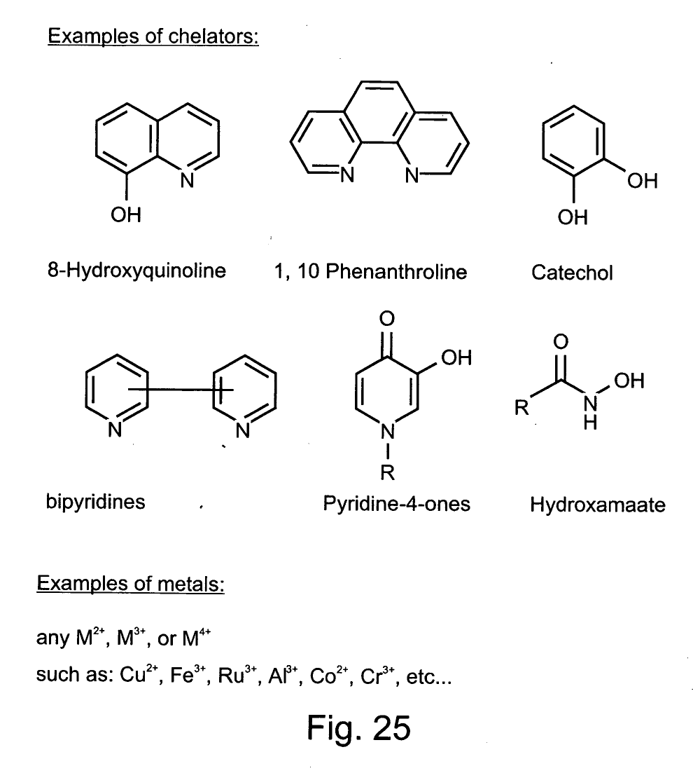

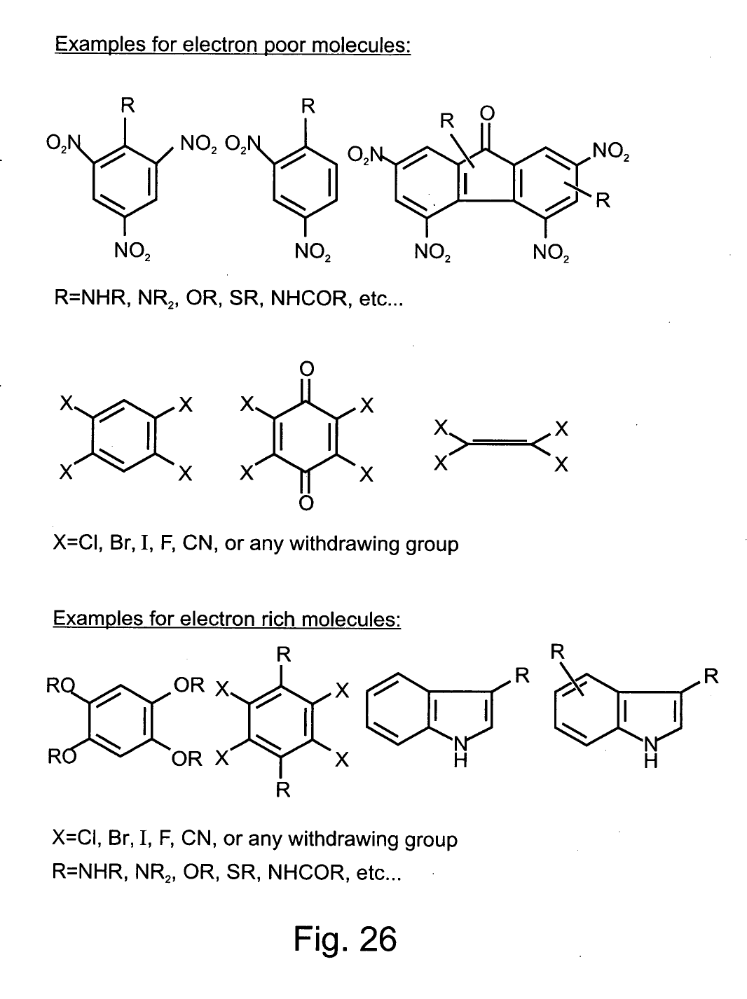

As used herein the phrase “coordinator ion or molecule” refers to a soluble entity (i.e., molecule or ion), which exhibits sufficient affinity (i.e., K_{D}<10⁻⁵) to the coordinating moiety and as such is capable of directing the composition of matter of this aspect of the present invention to form a non-covalent complex. Examples of coordinator molecules which can be used in accordance with the present invention include but are not limited to, avidin and derivatives thereof, antibodies, electron rich molecules, electron poor molecules and the like. Examples of coordinator ions which can be used in accordance with the present invention include but are not limited to, mono, bis or tri valent metals. FIG. 25 illustrates examples of chelators and metals which can be used as a coordinator ion by the present invention. FIG. 26 lists examples of electron rich molecules and electron poor molecules which can be used by the present invention. Methods of generating antibodies and antibody fragments as well as single chain antibodies are described in Harlow and Lane, Antibodies: A Laboratory Manual, Cold Spring Harbor Laboratory, New York, 1988, incorporated herein by reference; Goldenberg, U.S. Pat. Nos. 4,036,945 and 4,331,647, and references contained therein; See also Porter, R. R. [Biochem. J. 73: 119-126 (1959); Whitlow and Filpula, Methods 2: 97-105 (1991); Bird et al., Science 242:423-426 (1988); Pack et al., Bio/Technology 11:1271-77 (1993); and U.S. Pat. No. 4,946,778].

Preferably, the composition of this aspect of the present invention includes the coordinator ion or molecule.

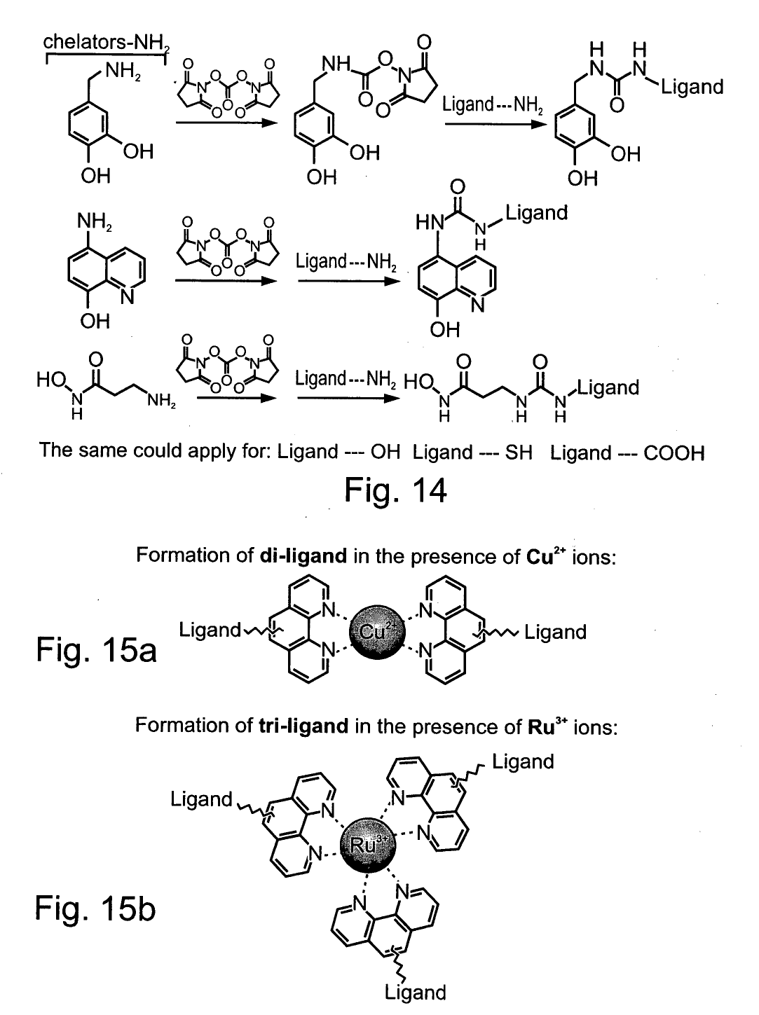

The ligand of this aspect of the present invention may be bound directly to the coordinating moiety, depending on the chemistry of the two. Measures are taken, though, to maintain recognition (e.g. affinity) of the ligand to the molecule of interest. When needed (e.g. steric hindrance), the ligand may be bound to the coordinating moiety via a linker. A general synthetic pathway for modification of representative chelators with a general ligand is shown in FIG. 14. Margherita et al. (1993) J. Biochem. Biophys. Methods 38:17-28 provides synthetic procedures which may be used to attach the ligand to the coordinating moiety of the present invention.

When the ligand and coordinating moiety bound thereto are both proteins (e.g. growth factor and epitope tag, respectively), synthesis of a fusion protein can be effected by molecular biology methods (e.g. PCR) or biochemical methods (solid phase peptide synthesis).

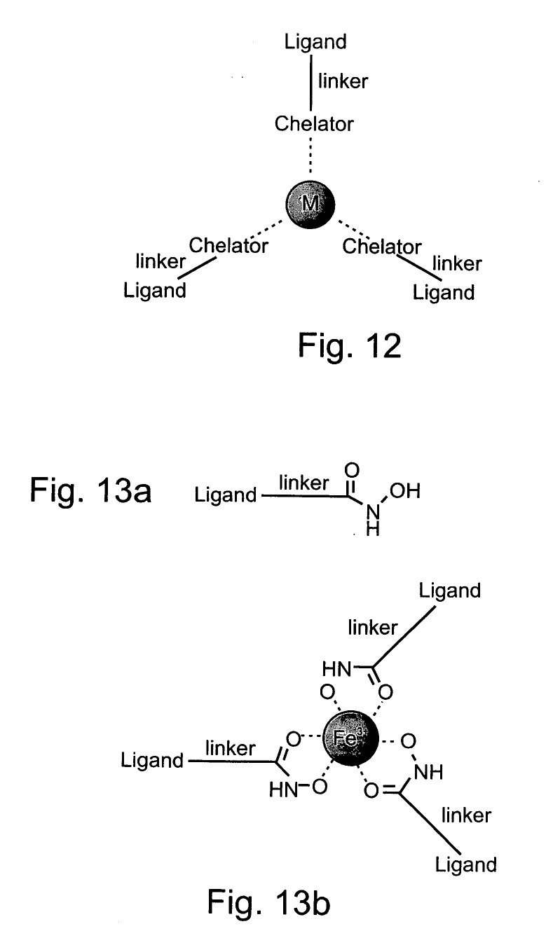

Complexes of the present invention be of various complexity levels, such as, monomers (see FIGS. 12 and 13a-b depicting a three ligand complex), dimers, polymers (see FIGS. 23a-b depicting formation of a polymer via a combined linker as described in Example 3 of the Examples section), sheets (see FIG. 24 in which sheets are formed when a single surface exposed Trp residue of a target molecule forms electron rich/poor relations with a TNB---TNB entity) and lattices which may form three dimensional (3D) structures (such as when more than one surface exposed Trp residues form electron rich/poor relations). It is well established that the higher complexity of the complex the more rigid is the structure enabling use thereof in crystallization procedures as further described hereinbelow. Furthermore, large complexes will phase separate more rapidly, negating the use of further centrifugation steps.

It will be appreciated that in cases where the composition of the present invention is utilized for purification of a target molecule/cell (see below for further description), the ligand is selected such that the target molecule/cell is uniformly bound to the complex. For example, the ligand can be selected such that the target molecule/cell bound by the complex is only associated with a single ligand molecule of the complex or with a predetermined number of ligand molecules. As is further described below, such uniform association between ligand and target molecule/cell ensures that purification of the target from the complex is uniform, i.e. that a single elution step releases substantially all of the complex-bound target.

Examples of ligand configuration which enable such uniform binding of the target molecule/cell, include: peptides (i.e., cyclic or linear), Protein A or G or L, antibodies, lectines (e.g., concanavalin A from Jack bean, Jacalin from Jack fruit), various dyes (e.g., Cibacron Blue 3GA) and aptamers.

The compositions of the present invention can be packed in a purification kit which may include additional buffers and additives, as described hereinbelow. It will be appreciated that such kits may include a number of ligands for purifying a number of molecules from a single sample. However, to simplify precipitation (e.g. using the same reaction buffer, temperature conditions, pH and the like) and further purification steps, the coordinating moieties and coordinator ions or molecules are selected the same.

As mentioned hereinabove, the compositions of the present invention may be used to purify a molecule or cell of interest from a sample.

Thus, according to another aspect of the present invention there is provided a method of purifying a molecule of interest.

As used herein the term “purifying” refers to at least separating the molecule of interest from the sample by changing its solubility upon binding to the composition of the present invention and precipitation thereof (i.e., phase separation).

The method of this aspect of the present invention is effected by contacting a sample including the molecule of interest with a composition of the present invention and collecting a precipitate which includes a complex formed from the composition-of-matter of the present invention and the molecule of interest, thereby purifying the molecule of interest.

As used herein the term “sample” refers to a solution including the molecule of interest and possibly one or more contaminants (i.e., substances that are different from the desired molecule of interest). For example when the molecule of interest is a secreted recombinant polypeptide, the sample can be the conditioned medium, which may include in addition to the recombinant polypeptide, serum proteins as well as metabolites and other polypeptides, which are secreted from the cells. When the sample includes no contaminants, purifying refers to concentrating.

In order to initiate purification, the composition-of-matter of the present invention is first contacted with the sample. This is preferably effected by adding the ligand attached to the coordinating moiety to the sample allowing binding of the molecule of interest to the ligand and then adding the coordinator ion or molecule to allow complex formation and precipitation of the molecule of interest. In order to avoid rapid formation of complexes (which may result in the entrapment of contaminants) slow addition of the coordinator to the sample while stirring is preferred. Controllable rate of precipitation can also be achieved by adding free coordinating entity (i.e., not bound to the ligand), which may also lead to the formation of smaller complexes which may be beneficial in a variety of applications such as for the formation of immunogens, further described hereinbelow.

Once the complex described above is formed (seconds to hours), precipitation of the complex may be facilitated by centrifugation (e.g. ultra-centrifugation), although in some cases (for example, in the case of large complexes) centrifugation is not necessary.

Depending on the intended use the molecule of interest, the precipitate may be subjected to further purification steps in order to recover the molecule of interest from the complex. This may be effected by using a number of biochemical methods which are well known in the art. Examples include, but are not limited to, fractionation on a hydrophobic interaction chromatography (e.g. on phenyl sepharose), ethanol precipitation, isoelectric focusing, reverse phase HPLC, chromatography on silica, chromatography on heparin sepharose, anion exchange chromatography, cation exchange chromatography, chromatofocusing, SDS-PAGE, ammonium sulfate precipitation, hydroxylapatite chromatography, gel electrophoresis, dialysis, and affinity chromatography (e.g. using protein A, protein G, an antibody, a specific substrate, ligand or antigen as the capture reagent).

It will be appreciated that simple addition of clean reaction solution (e.g. buffer) may be added to the precipitate to elute low affinity bound impurities which were precipitated during complex formation.

It will be further appreciated that any of the above-described purification procedures may be repetitively applied on the sample (i.e., precipitate) to increase the yield and or purity of the target molecule.

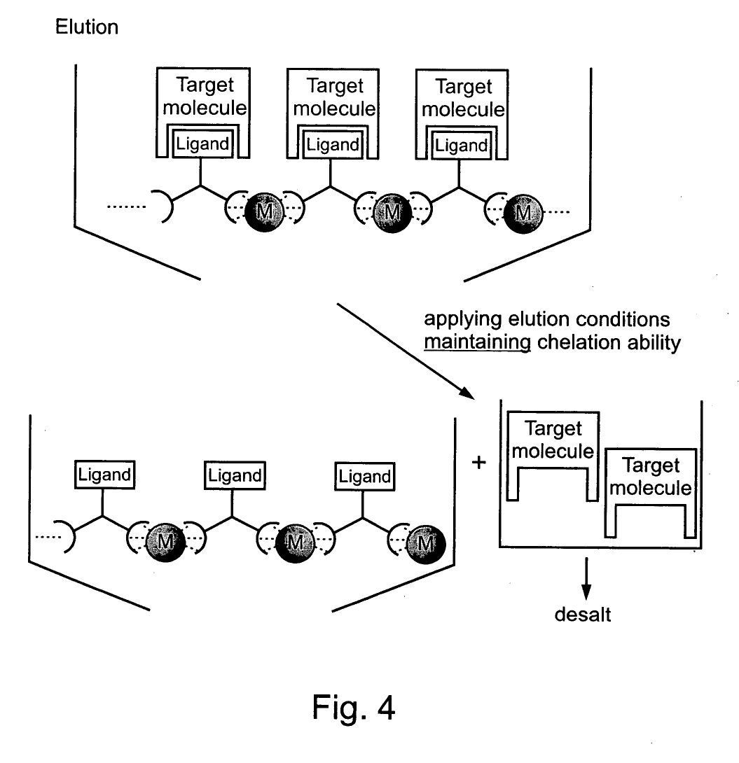

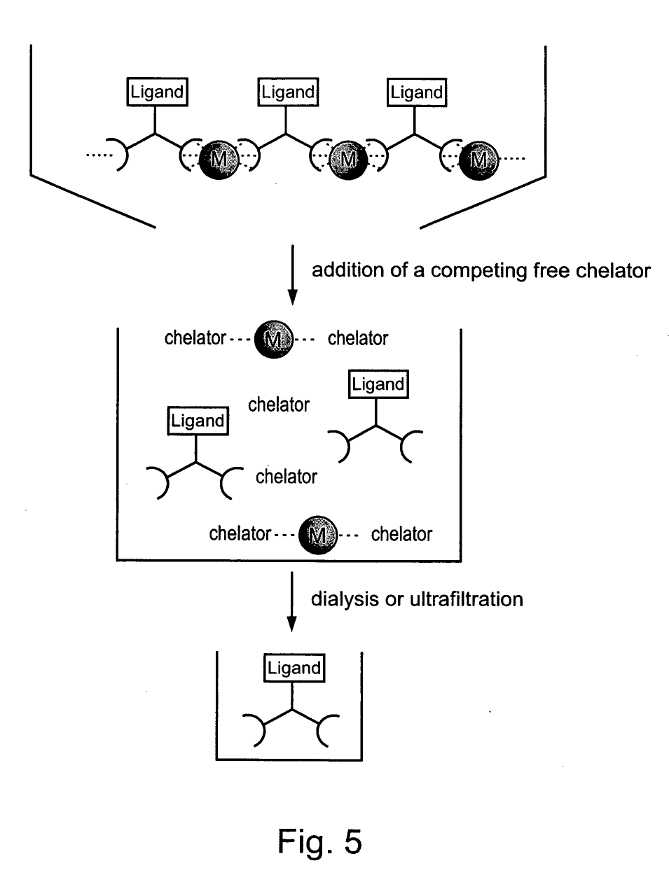

Preferably, the composition of matter and coordinator ion or molecule are selected so as to enable rapid and easy isolation of the target molecule from the complex formed. For example, the molecule of interest may be eluted directly from the complex, provided that the elution conditions employed do not disturb binding of the coordinating moiety to the coordinator (see FIGS. 4-5). For example, when the coordinating moiety used in the complex is a chelator, high ionic strength may be applied to elute the molecule of interest, since it is well established that it does not effect metal-chelator interactions. Alternatievly, elution with chaotropic salt may be used, since it has been shown that metal-chelator interactions are resistant to high salt conditions enabling elution of the target molecule at such conditions [Porath (1983) Biochemistry 22:1621-1630].

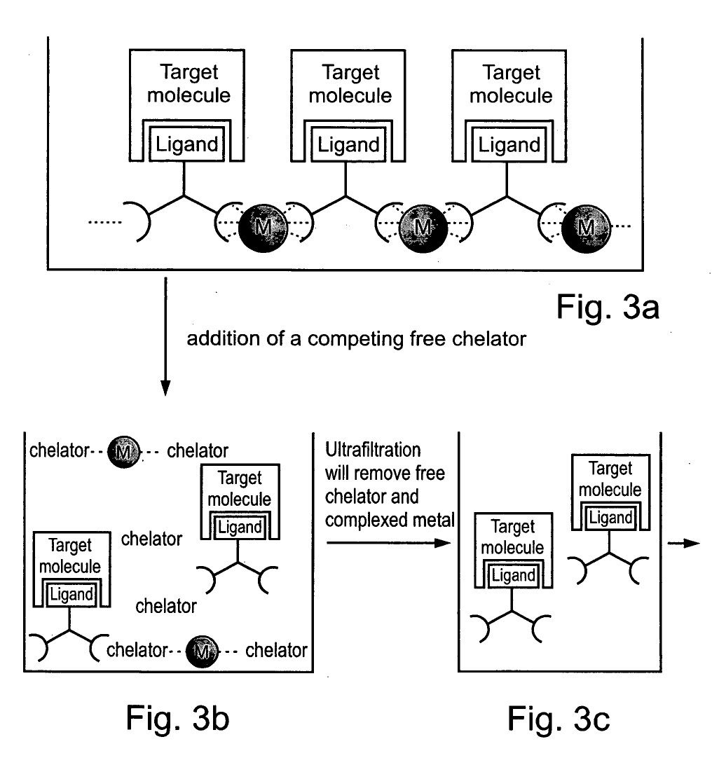

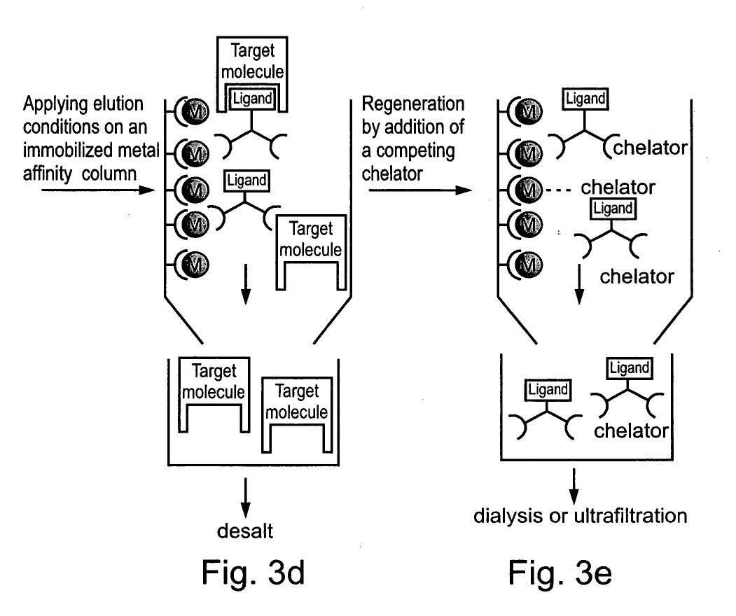

The complex can be re-solubilized by the addition of free (unmodified) chelator (i.e., coordinating moiety), which competes with the coordinator metal (FIG. 3). Ultrafiltration or dialysis may be used, thereafter, to remove most of the chelated metal and the competing chelator. The solubilized complex (i.e., molecule of interest:ligand-coordinating moiety) can then be loaded on an immobilized metal affinity column [e.g. iminodiacetic acid (IDA) and nitrilotriacetic acid (NTA)]. It will be appreciated that when high affinity chelators are used (e.g. catechol), measures are taken to use immobilized metal affinity ion column modified with the same or with other chelator having similar binding affinities toward the immobilized metal, to avoid elution of the ligand:chelator agent from the column instead of binding to it.

Application of suitable elution conditions will result in the elution of the target molecule keeping the ligand-coordinating moiety bound to the column. A final desalting procedure may be applied to obtain the final product.

Regeneration of the ligand-coordinating moiety is of high economical value, since synthesis of such a fusion molecule may contribute most of the cost and labor involved in the methodology described herein. Thus, for example, regeneration of the ligand-coordinating moiety can be achieved by loading the above-described column with a competing chelator or changing column pH followed by ultrafiltration that may separate between the free chelator and the desired ligand-coordinating moiety.

The Examples section which follows provides specific examples of binding/elution protocols which can be used with the present invention. It will be appreciated however, that the described parameters can be varied according to the immobilized target and purity needs.

Thus, several binding/washing/elution/regeneration parameters can be utilized by the present invention, including:

(i) diverse pH values (e.g. pH=2-10);

(ii) presence of different salts (other than NaCl) or in combination and in various concentrations (e.g. 1 μM-5M);

(iii) presence or absence of free metal chelator/s or combinations of chelators (e.g. imidazole catechol, His and catechol, His and EDTA, phosphate and EGTA, Citrate and 1,10-phenanthroline, etc.);

(iv) different buffers (other than sodium phosphate) i.e. Tris, Citrate, PBS, Gly at various concentrations and pH values;

(v) presence or absence of radical scavengers;

(vi) addition of divalent, trivalent or tetravalent metals (Ca2+, Mg2+, Mn2+, Co2+, Al3+, Th4+);

(vii) various temperature ranges (other than 0-4° C.);

(viii) various incubation times, e.g. the binding of the modified ligand to the target may change when that target is at low concentration or binding between the two is relatively weak, ligand will be required when the target is at low concentration or when the affinity of the ligand toward the target is low;

(ix) different sequences of additions, for example, addition of salt, then ligand then free chelator, then metal, or, addition of salt, then ligand, then metal, then free chelator;

(x) use of ligands modified with chelators other than catechol (e.g. hydroxy quinoline derivatives);

(xi) modification of a ligand with a chelator (for example) having different leaving groups (e.g. catechol-meleimide, catechol-iodacetamide, catechol-chloroactyl); and/or

(xii) with or without use of detergents (e.g. SDS, Triton)

The above-described purification methodology can be applied for the isolation of various recombinant and natural substances which are of high research or clinical value such as recombinant growth factors and blood protein products (e.g. von Willebrand Factor and Factor VIII which are therapeutic proteins effective in replacement therapy for von Willebrand's disease and Hemophilia A, respectively).

As mentioned hereinabove, the compositions of the present invention may also be used to isolate particular populations of cells, antigens, viruses, plasmids and the like. The following section exemplifies use of the present invention in such applications.

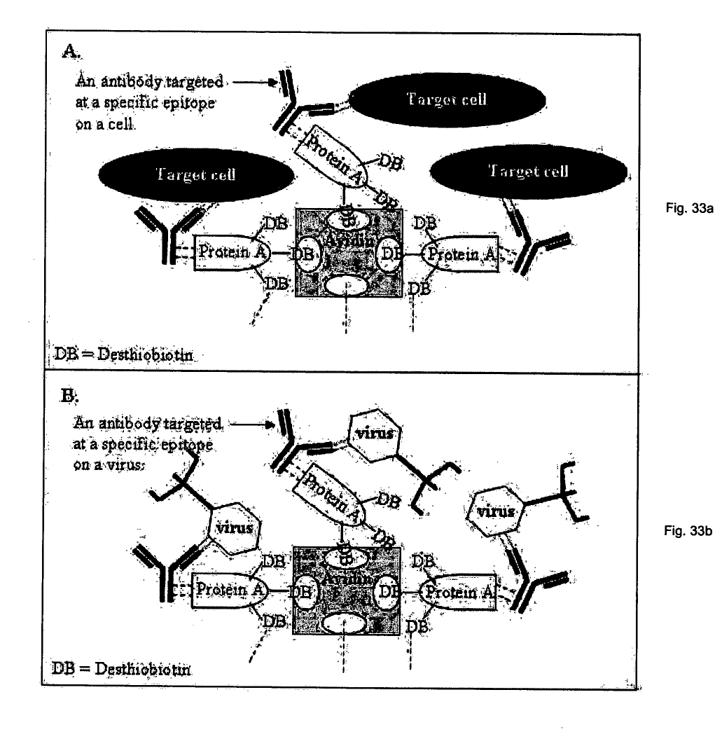

Positive selection of cells The present invention can be utilized to isolate cancer cells or stem cells which possess unique surface markers. For example, cells displaying CD34 and CD105 [see Pierelli (2001) Leuk. Lymphoma 42(6):1195-206]) can be isolated by incubation of a cell suspension with a mAb directed at an epitope on the target cell, followed by addition of desthiobiotinylated protein A (which could be added together with the mAb itself). The target cell-mAb-modified protein A (or G or L) complex (also referred to herein as the Precipitating complex) would precipitate the target cell upon addition of free avidin. The supernatant will be discarded while the pellet containing the target cell would be either directly used; agitated to free bound cells from the precipitate; incubated in the presence of a competing molecule (e.g. peptide) which would release the target cell by competing with the epitope of the cell on binding to the mAb; or incubated in the presence biotin (or its analogues) for partial or total dissolution of the pellet thereby, enabling an effective cell release (for further detail see FIG. 33).

Negative selection of cells the precipitating complex described above can be used along with a single mAb or several mAbs targeted at non-relevant cells in order to precipitate non-target cells and form a supernatant containing enriched medium of target cells.

Specific antigen precipitation the precipitating complex described above can be utilized with a target antigen known to bind to an mAb/s forming a part of the complex.

Depletion of viruses the precipitating complex described above can be used with virus or viruses containing an epitope known to bind to an mAb/s forming a part of the complex.

Precipitation of DNA/RNA-protein complexes the precipitating complex described above can utilize an mAb/s which can bind DNA/RNA-protein.

Plasmid purification the Precipitating complex described above can utilize an antibody which binds directly to a plasmid.

It will be appreciated that an antibody or mAb utilized by the precipitating complex could be used as “modification platform”, into which ligands or nucleotide sequences are covalently attached. The modified antibody could then be utilized for all the above described applications. Such an approach will circumvent the need for antibodies specific to target biomolecules.

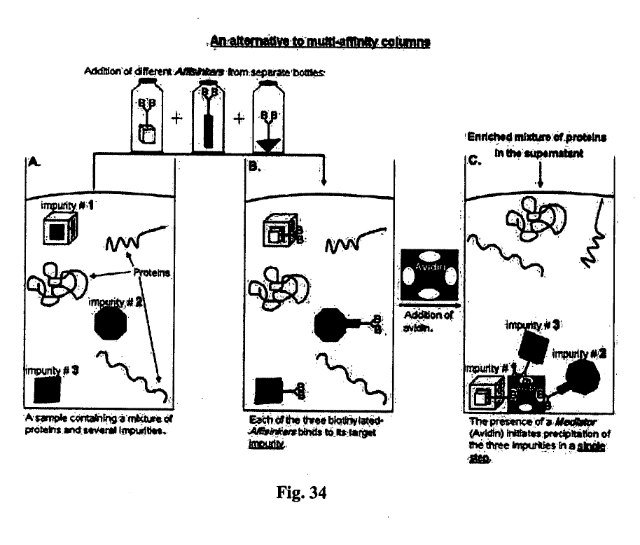

The present compositions can also be utilized for reducing contamination or background. For example, several ligands may be modified with the same coordinating entity (e.g. biotin) and incubated in a medium containing impurities known to bind to the modified ligands. Removal of impurities will be initiated by addition of free avidin (for example), and the enriched supernatant could be used for further applications (see FIG. 34 for further detail).

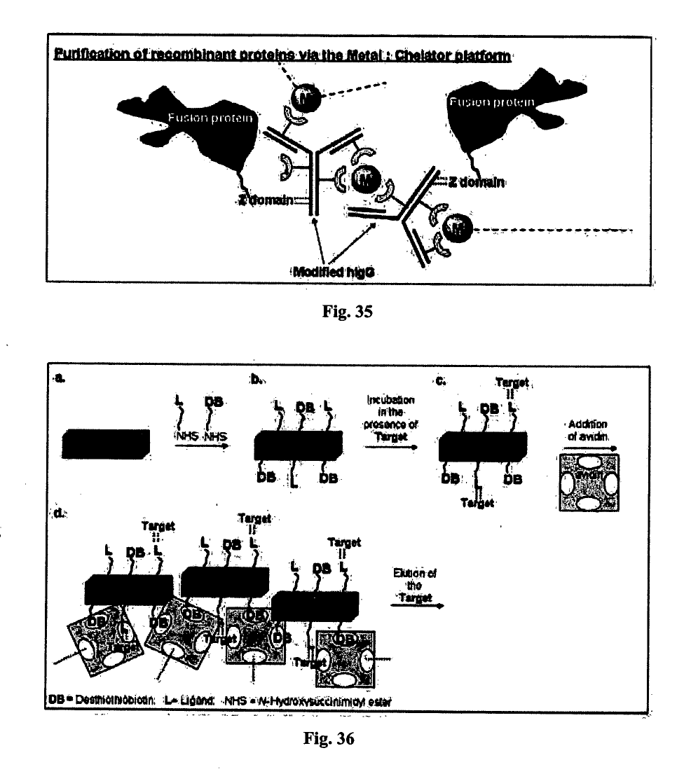

Purification of recombinant proteins possessing fusion partners such as the Z (or ZZ) domain of Protein A could be purified in the presence of a modified human IgG (hIgG) to which the Z domain binds specifically, followed by addition of an appropriate transition metal which would generate insoluble macro-complexes containing the fusion protein (see FIG. 35 for further detail). These macro-complexes would precipitate while impurities left soluble in the supernatant will be excluded. The same could be applied to other recombinant proteins with the following fusion partners:

(i) Recombinant protein—ABP (Albumin Binding Protein of Protein G) and a modified HSA (Human Serum Albumin).

(ii) Recombinant protein—MBP (E. coli Maltose Binding Protein) and a modified amylose.

(iii) Recombinant protein—GST and a modified Glutathione.

(iv) Recombinant protein—FLAG peptide and a modified mAb M1 or mAb M2.

The present invention can also utilize non-immobilized multivalent ligands (NML) which can be generated via covalent linking of a protein (e.g. ovalbumin) with any ligand (e.g. Fluorescein) and a complexing entity (e.g. desthiobiotin). The modified protein (see FIG. 36 for further detail) serves as the MNL since it is capable of interacting specifically with a Target molecule (FIG. 36 step b) and be further precipitated upon addition of an appropriate mediator entity (e.g. free avidin) (FIG. 36 step c) which will interconnect modified ovalbumins (FIG. 36 step d). Thus, specific precipitation is initiated in the presence of avidin whereas impurities are left soluble in the supernatant and are excluded. The Target is then eluted from the precipitate (i.e. pellet) under conditions favoring dissociation of the Target rather than dissociation of the [ovalbumin-desthiobiotin:avidin] multi-complex (FIG. 36 step d)

An efficient elution may be accomplished by using networks with lower degree of complexity (e.g. a network which includes larger holes). These could be generated by an avidin solution containing also bis, tris or multi avidin complexes that were cross-linked prior to their incubation with bis, tris or multi biotin moieties. (or their derivatives), via modification of the ligand with a complexing (coordinating) entity having extended spacer arms or by using avidin molecules that were incubated with free biotin prior to their use as a coordinator molecule. Similarly, free biotin may be present before the addition of avidin (see Example 7).

It is well established that due to shortage in human organs, in-vitro organogenesis is emerging as an optimal substitute. To this end, stem cells which are capable of differentiating to any desired cell lineage must be isolated. Thus, for example, to isolate hematopoietic stem/progenitor cells a number of ligands may be employed which bind to surface markers which are unique to this cell population, such as CD34 and CD105 [see Pierelli (2001) Leuk. Lymphoma 42(6):1195-206].

Another example is the isolation of erythrocytes using lectin ligands, such as concanavalin A [Sharon (1972) Science 177:949; Goldstein (1965) Biochemistry 4:876].

Viral cell isolation may be effected using various ligands which are specific for viral cells of interest [see www.bdbiosciences.com/clontech/archive/JAN04UPD/Adeno-X.shtml].

Specifically, retroviruses may be isolated by the compositions of the present invention which are designed to include a heparin ligand [Kohleisen (1996) J Virol Methods 60(1):89-101].

Cell isolation using the above-described methodology may be effected with preceding steps of sample de-bulking which is effected to isolate cells based on cell density or size (e.g. centrifugation) and further steps of selective cell-enrichment (e.g. FACS).

On top of their purifying capabilities, the compositions of the present invention may also be used to deplete a sample from undesired molecules or cells.

This is effected by contacting the sample including the undesired target molecule or cell of interest with the composition of the present invention such that a complex is formed (described above) and removing the precipitate. The clarified sample is the supernatant.

This method have various uses such as in depleting tumor cells from bone marrow samples, depleting B cells and monocytes for the isolation and enrichment of T cells and CD8⁺ cells or CD4⁺ cells from peripheral blood, spleen, thymus, lymph or bone marrow samples, depleting pathogens and unwanted substances (e.g. prions, toxins) from biological samples, protein purification (e.g. depleting high molecular weight proteins such as BSA) and the like.

As mentioned hereinabove multiple ligands may be employed for the depletion of a number of targets from a given sample such as for the removal of highly abundant proteins from biological fluids (e.g. albumin, IgG, anti-trypsin, IgA, transferrin and haptoglobin, see http://www.chem.agilent.com/cag/prod/ca/51882709small.pdf).

The unique properties of the novel compositions of the present invention provide numerous advantages over prior art precipitation compositions (e.g. smart polymers), some of these these advantages are summerized infra.

(i) Low cost purification; the present methodology does not rely upon sophisticated laboratory equipment such as HPLC, thereby circumventing machine maintenance and operating costs.

(ii) Easy up scaling; the present methodology is not restricted by limited capacity of affinity columns having diffusion limitations. Essentially, the amount of added precipitating complex is unlimited.

(iii) Mild precipitation process; averts limitations resulting from substantial changes in pH, ionic strength or temperature.

(iv) Uniform purification process; in the case of a complex having a ligand capable of uniform (e.g. monovalent) interactions with the target (i.e. a predetermined number of ligands per target or vice versa), uniform purification can be achieved under selected elution conditions since the target molecules/cells are uniformly bound to the complex.

(v) Control over the precipitation process; precipitation may be governed by, slow addition of an appropriate coordinator ion or molecule to the precipitation mixture; use of mono and/or multi-valent coordinators; use of coordinator ions or molecules with different affinities towards the coordinating moiety; addition of the non-immobilized free coordinating moieties to avoid non-specific binding and entrapment of impurities prior to, during or following formation of a non-covalent polymer, sheet or lattice [Mattiasson et al., (1998) J. Mol. Recognit. 11:211-216; Hilbrig and Freitag (2003) J. Chromatogr. B 790:79-90]; as well as by varrying temperature conditions. It is well established that various molecules exhibit lower solubility as the temperature decreases, therefore, controlling temperature conditions may regulate the rate and degree of precipitation. It will be appreciated, though, that low temperature conditions may lead to entrapment of impurities due to a fast precipitation process, while high temperature conditions may lead to low yields of the target molecule (e.g. denaturing temperatures). Thus measures are taken to achieve optimal temperature conditions, while considering the above parameters.

(vi) Reduced contamination background; contaminants cannot bind the coordinator entity and as such they cannot bind tightly to the non-covalent matrix, allowing their removal prior to the elution step. Furthermore, contaminations deriving from the ligand biological background (molecules which co-purified with the ligand) may become modified as well as the ligand itself [provided that the ligand and the contaminants share the same chemistry (e.g. both being proteins)], and might become part of the precipitating complex. Under suitable elution conditions, the target molecule will be recovered, while the modified contaminations will not.

(vii) Binding in homogenous solutions; it is well established that binding in homogeneous solution is more rapid and more effective than in heterogeneous phases such as in affinity chromatography [A C, Schneider et al., (1981) Ann. NY Acad. Sci. 369, 257-263; Lowe (2001) J. Biochem. Biophys. Methods 49, 561-574]. For example, high molecular mass polymers (used in AP) are known to form highly coiled and viscous structures in solutions that hinder the access of incoming macromolecules such as the target molecules as in many affinity separation strategies. [Vaida et al., (1999) Biotechnol. Bioeng. 64:418].

(viii) No immobilization of the ligand—further described hereinabove.

(ix) Easy resolubilization of the complex; the complex is generated by non-covalent interactions.

(x) Sanitizing under harsh conditions; the composition is not covalently bound to a matrix and as such can be removed from any device, allowing application of sanitizing conditions to clean the device (column) from non-specifically bound impurities.

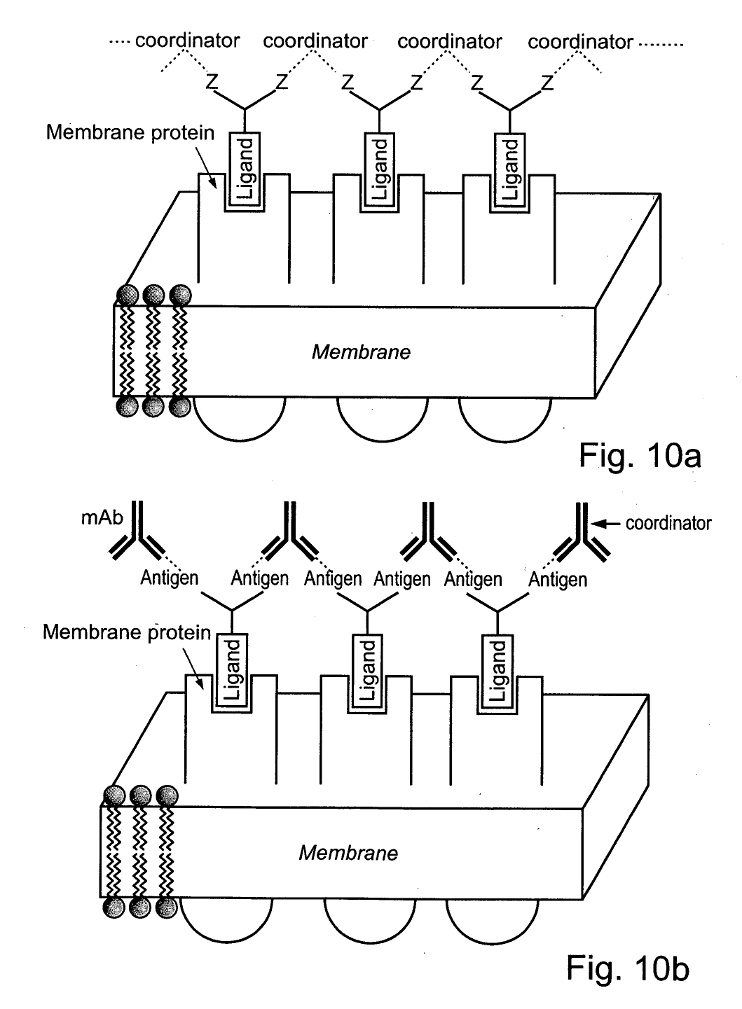

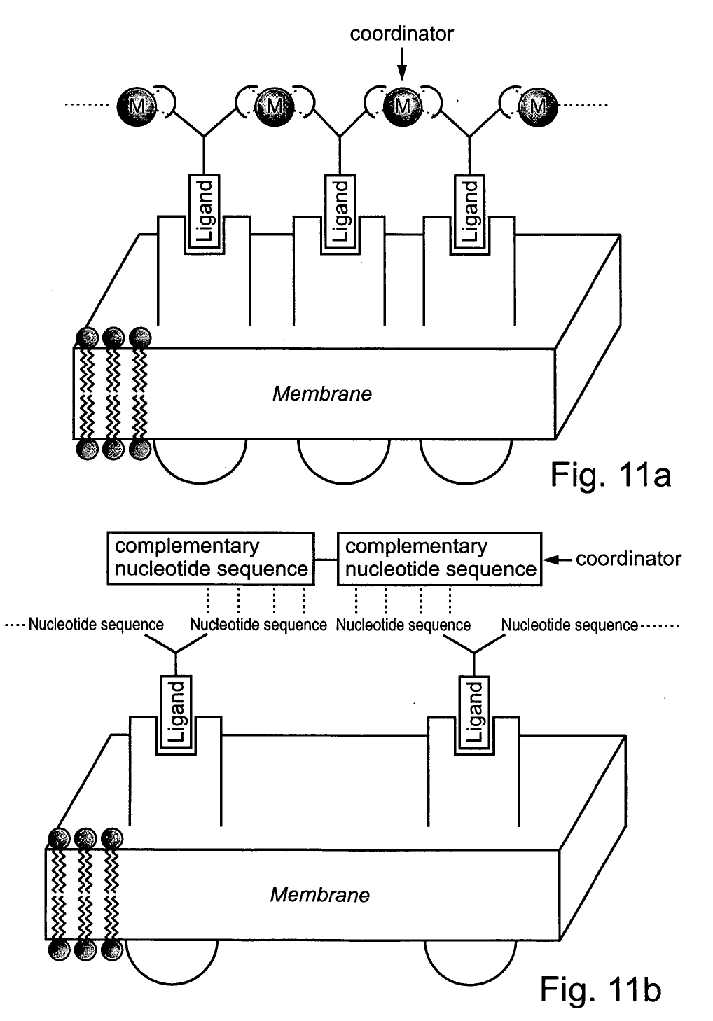

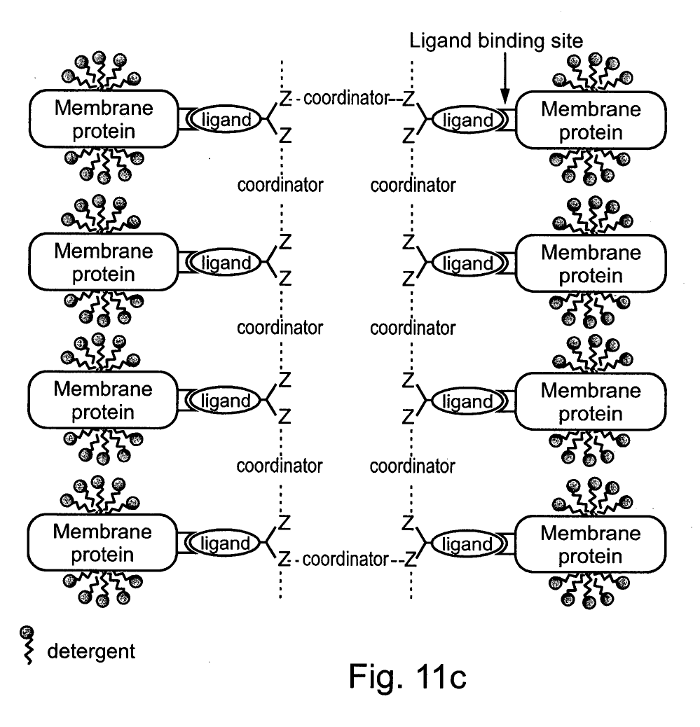

The ability of the compositions of the present invention to arrange molecules of interest in ordered complexes such as in dimers, trimers, polymers, sheets or lattices also enables use thereof in facilitating crystallization of macromolecules such as proteins, in particular membraneous proteins. As is well known in the art, a crystal structure represents ordered arrangement of a molecule in a three dimensional space. Such ordered arrangement can be egenerated by reducing the number of free molecules in a given space (see FIGS. 10a-b and 11a-c).

Thus, according to yet another aspect of the present invention there is provided a composition for crystallizing a molecule of interest.

As used herein the term “crystallizing” refers to the solidification of the molecule of interest so as to form a regularly repeating internal arrangement of its atoms and often external plane faces.

The composition of this aspect of the present invention includes at least one ligand capable of binding the molecule of interest, wherein the ligand is attached to at least one coordinating moiety; and a coordinator capable of non-covalently binding the at least one coordinating moiety, wherein the at least one coordinating moiety and the coordinator are capable of forming a complex when co-incubated and whereas the composition is selected so as to define the relative spatial positioning and orientation of the molecule of interest when bound thereto, thereby facilitating formation of a crystal therefrom under inducing crystallization conditions.

It will be appreciated that the use of covalent multi ligand complexes has been previously attempted in the crystallization of soluble proteins [Dessen (1995) Biochemistry 34:4933-4942; Moothoo (1998) Acta. Cryst. D54 1023-1025; Bhattacharyya (1987) J. Biol. Chem. 262:1288-1293]. However, synthesis of multi-ligand complexes which have more than two ligands per molecule is technically difficult and expensive; Furthermore, the three-dimensional structure of the target protein should be known in advance to synthesize multi ligand complexes which have the optimal distance between the ligands to bind enough target molecules to occupy all target binding sites in the multi-ligand complex, as such, these ligands were never used for the crystallization of membrane proteins.

The present invention circumvents these, by synthesizing only the basic unit in the non-covalent multi-ligand, (having the general structure of: Ligand-coordinating moiety) which is far easier to achieve, faster and cheaper. This basic unit, would form non-covalent tri-ligand only by adding the multi valent coordinator ion or molecule. Thus, a single synthesis step is used to form di, tri, tetra or higher multi ligands that may be used for crystallization experiments.

In order to produce crystals of a molecule of interest (preferably of membrane proteins) the compositions of the preset invention are contacted with a sample, which includes the molecule of interest preferably provided at a predetermined purity and concentration.

Typically, the crystallization sample is a liquid sample. For example, when the molecule of interest is a membrane protein, the crystallization sample, according to this aspect of the present invention, is a membrane preparation. Methods of generating membrane preparations are described in Strategies for Protein Purification and Characterization—A Laboratory Course Manual” CSHL Press (1996).

Once the molecule of interest is bound to the composition of the present invention, such that its relative spatial positioning and orientation are well defined, the sample is subjected to suitable crystallization conditions. Several crystalization approaches which are known in the art can be applied to the sample in order to facilitate crystalization of the molecule of interest. Examples of crystallization approaches include, but are not limited to, the free interface diffusion method [Salemme, F. R. (1972) Arch. Biochem. Biophys. 151:533-539], vapor diffusion in the hanging or sitting drop method (McPherson, A. (1982) Preparation and Analysis of Protein Crystals, John Wiley and Son, New York, pp 82-127), and liquid dialysis (Bailey, K. (1940) Nature 145:934-935).

Presently, the hanging drop method is the most commonly used method for growing macromolecular crystals from solution; this approach is especially suitable for generating protein crystals. Typically, a droplet containing a protein solution is spotted on a cover slip and suspended in a sealed chamber that contains a reservoir with a higher concentration of precipitating agent. Over time, the solution in the droplet equilibrates with the reservoir by diffusing water vapor from the droplet, thereby slowly increasing the concentration of the protein and precipitating agent within the droplet, which in turn results in precipitation or crystallization of the protein.

Crystals obtained using the above-described methodology, have a resolution of preferably less than 3 Å, more preferably less than 2.5 Å, even more preferably less than 2 Å.

Compositions of the present invention may have evident utility in assaying analytes from complex mixtures such as serum samples, which may have obvious diagnostic advantages.

Thus, the present invention envisages a method of detecting predisposition to, or presence of a disease associated with a molecule of interest in a subject.

An example of a disease which is associated with a molecule of interest is prostate cancer which may be detected by the presence of prostate specific antigen [PSA, e.g. >0.4 ng/ml, Boccon-Gibod Int J Clin Pract. (2004) 58(4):382-90].

The compositions of the present invention are contacted with a biological sample obtained from the subject whereby the level of complex formation including the molecule of interest is indicative of predisposition to, or presence of the disease associated with the molecule of interest in the subject.

As used herein the phrase “biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, milk, blood cells, tumors, neuronal tissue, organs, and also samples of in vivo cell culture constituents.

To facilitate detection and quantification of the molecule of interest in the complexes, the biological sample or the composition is preferably labeled (e.g. fluorescent, radioactive labeling).

Compositions of the present invention may also be utilized to qualify and quantify substances present in a liquid or gaseous samples which may be of great importance in clinical, environmental, health and safety, remote sensing, military, food/beverage and chemical processing applications.

Abnormal protein interaction governs the development of many pathogenic disorders. For example, abnormal interactions and misfolding of synaptic proteins in the nervous system are important pathogenic events resulting in neurodegeneration in various neurological disorders. These include Alzheimer's disease (AD), Parkinson's disease (PD), and dementia with Lewy bodies (DLB). In AD, misfolded amyloid beta peptide 1-42 (Abeta), a proteolytic product of amyloid precursor protein metabolism, accumulates in the neuronal endoplasmic reticulum and extracellularly as aggregates (i.e., plaques). The compositions of the present invention can be used to disturb such macromolecular complexes to thereby treat such disorders.

Methods of administration and generation of pharmaceutical compositions are described by, for example, Fingl, et al., (1975) “The Pharmacological Basis of Therapeutics”, Ch. 1 p. 1.

The compositions of the present invention can be included in a diagnostic or therapeutic kits. For example, compositions of a specific disease can be packaged in a one or more containers with appropriate buffers and preservatives and used for diagnosis or for directing therapeutic treatment.

Thus, the ligand and coordinating moiety can be placed in one container and the coordinator molecule or ion can be placed in a second container. Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

A number of methods are known in the art for enhancing the immunogenic potential of antigens. For example, hapten carrier conjugation which involves cross-linking of the antigenic molecule (e.g. peptides) to larger carriers such as KLH, BSA thyroglobulin and ovalbumin is used to elevate the molecular size of the molecule, a parameter known to govern immunogenicity [see Harlow and Lane (1998) A laboratory manual Infra]. However, covalent cross-linking of the antigenic molecule leads to structural alterations therein, thereby limiting antigenic presentation. Non-covalent immobilization of the antigenic molecule to various substrates have been attempted to circumvent this problem [Sheibani Frazier (1998) BioTechniques 25:28]. Accordingly, compositions of the present invention may be used to mediate the same.

Thus, the present invention also envisages a method of enhancing immunogenicity of a molecule of interest using the compositions of the present invention. As used herein the term “immunogenicity” refers to the ability of a molecule to evoke an immune response (e.g. antibody response) within an organism.

The method is effected by contacting the molecule of interest with the composition of the present invention whereby the complex thus formed serves as an immunogen. Such a complex can be injected to an animal host to generate an immune response.

Thus, for example, to generate an antibody response, the above-described immunogenic composition is subcutaneously injected into the animal host (e.g. rabbit or mouse). Following 1-4 injections (i.e., boosts), serum is collected (about 14 weeks of first injection) and antibody titer is determined such as by using the above-described methods of analyte detection in samples, where the ligand is protein A for example. Alternatively or additionally, affinity chromatography or ELISA is effected.

It will be appreciated that the compositions of the present invention may have numerous other utilities, which are not distinctly described herein such as those utilities, which are attributed to affinity chromatography [see e.g. Wen-Chien and Kelvin (2004) Analytical Biochemistry 324:1-10].

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Synthesis of Non Covalent Multi Ligand Complexes Utilizing Chelator-Metal Complexes

The ability of chelators to bind metals, with different specificities and affinities is well described in the literature. To generate the non-covalent multi ligand complex of the present invention, a linker, (of a desired length) is modified to bind a specific ligand, and a chelator to generate the following general structure of: ligand----linker----chelator.

Then, by the addition of an appropriate metal, a non-covalent multi-ligand complex should be formed. (FIG. 12)

For example, a hydroxamate (which is a known Fe³⁺ chelator) derivative is synthesized (FIG. 13a) such that in the presence of Fe³⁺ ions, a non-covalent multi-ligand complex is formed (FIG. 13b). A general synthetic pathway for modification of representative chelators with a general ligand is shown in FIG. 14. Such a synthesis can be similar to the one presented by Margherita et al., 1999 supra.

The utilization of chelators for the preparation of a non-covalent multi-ligand complex, may have an additional advantage which arises from the ability of some chelators to bind different metals with different stochiometries, as in the case of [1,10-phenanthroline]₂-Cu²⁺, or [1,10-phenanthroline]₃-Ru³⁺ [Onfelt et al., (2000) Proc. Natl. Acad. Sci. USA 97:5708-5713].

This phenomenon can be utilized for formation of di (FIG. 15a) and tri (FIG. 15b) non-covalent multi-ligand complexes, utilizing the same: ligand----linker----chelator derivative.

### Example 2

### Synthesis of Non-Covalent Multi Ligand Complexes Utilizing Electron Rich-Poor Complexes

Electron acceptors form molecular complexes readily with the “π excessive” heterocyclic indole ring system. Indole picric acid was the first complex of this type to be described nearly 130 years ago [Baeyer, and Caro, (1877) Ber. 10:1262] and the same electron acceptor was used a few years later to isolate indole from jasmine flower oil. Picric acid had since been used frequently for isolating and identifying indoles as complexes from reaction mixtures. Later, 1,3,5-trinitro benzene was introduced as a complexing agent and often used for the same purpose [Merchant, and Salagar, (1963) Current Sci. 32:18]. Other solid complexes of indoles have been prepared with electron acceptors such as: styphnic acid [Marion, L., and Oldfield, C. W., (1947) Cdn. J. Res. 25B 1], picryl halides [Triebs, W., (1961) Chem. Ber. 94:2142], 2,4,5,7-tetranitro-9-fluorenone [Hutzinger, O., and Jamieson, W. D., Anal. Biochem. (1970) 35, 351-358], and with 1-fluoro-2,4-dinitorbenzene and 1-chloro-2,4-dinitorbenzene [Elguero et al., (1967) Anals Real Soc. Espan. Fis. Quim. (Madrid) ser. B 63, 905 (1967); Wilshire, J. F. K., Australian J. Chem. 19, 1935 (1966)].

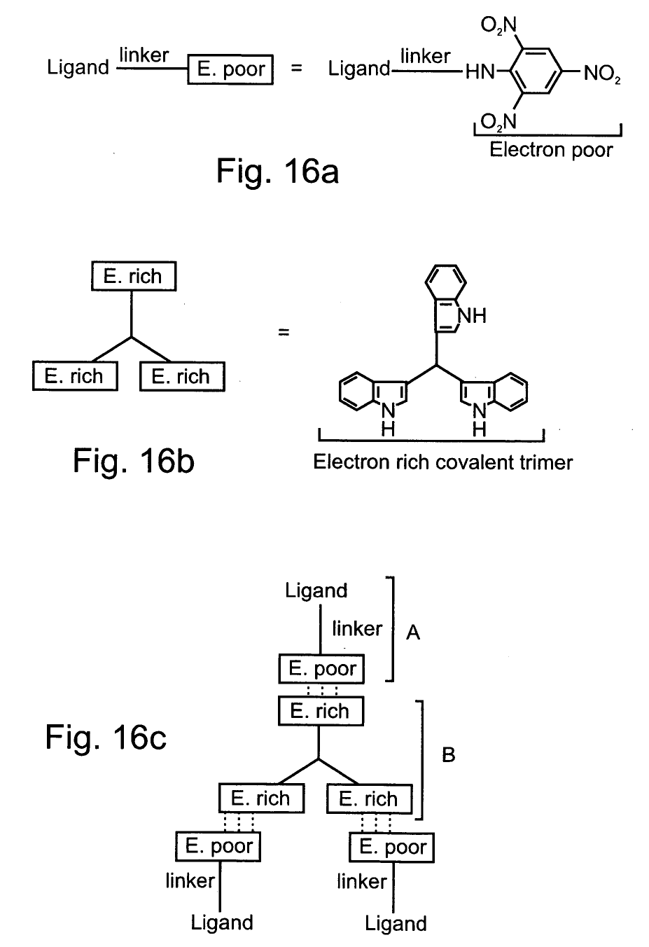

FIG. 16a, illustrates one example of a ligand----linker----electron poor (E. poor) derivative, and FIG. 16b, presents an example of an electron rich covalent trimer that could be used. It is expected, that by mixing together the trinitrobenzene (FIG. 16a) and the indole (FIG. 16b) derivatives, a multi-ligand complex will be formed (FIG. 16c). It will be appreciated that the reverse complex could be synthesized as well, i.e., a ligand derivative with an electron rich moiety, and an electron poor covalent trimer.]

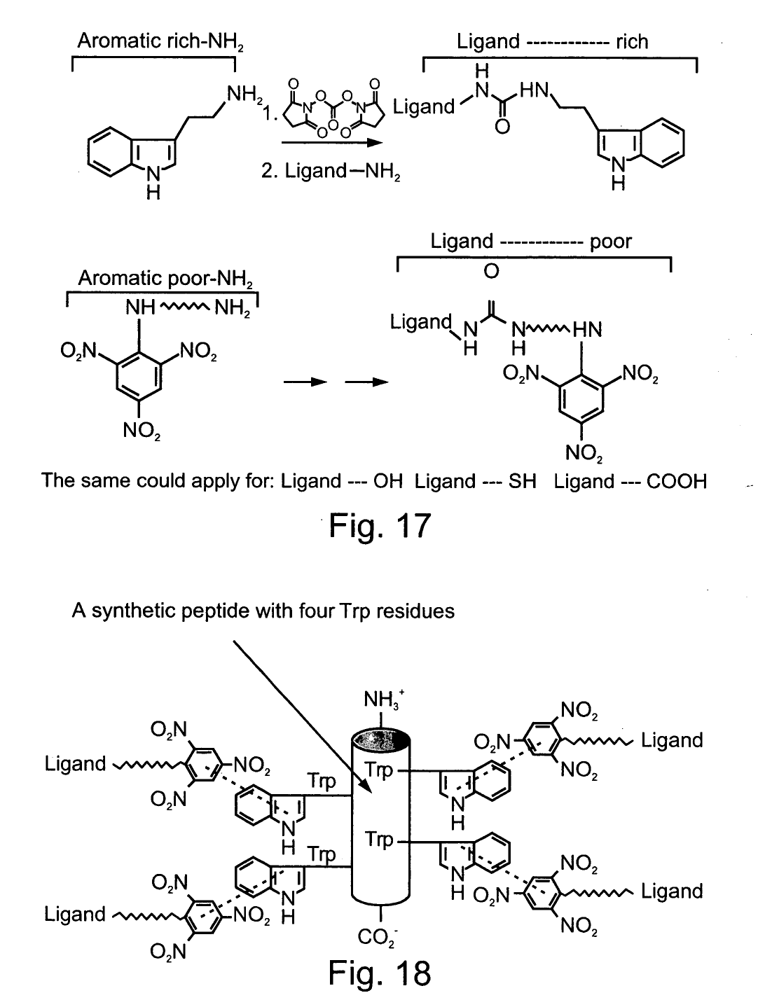

A possible synthetic pathway for the preparation of the above ligand derivatives is shown in FIG. 17.

Synthetic peptides (or any peptide) containing Trp residues (or any other electron rich or poor moieties) may also be of use for the preparation of non-covalent multi ligand complexes. FIG. 18 shows an example of a synthetic peptide with four Trp residues (four electron rich moieties) that can be formed, a tetra-non-covalent-ligand in the presence of a ligand derivative modified with an electron poor moiety (trinitrobenzene).

### Example 3

### Synthesis of Non-Covalent Multi Ligand Complexes Utilizing a Combination of Electron Rich-Poor and Chelator-Metal Relationships

One can combine the two complexing abilities as described in Examples 1 and 2 above, so as to form non-covalent multi ligand complexes. An example of the general structure of such a non-covalent multi ligand complex is shown in FIG. 19.

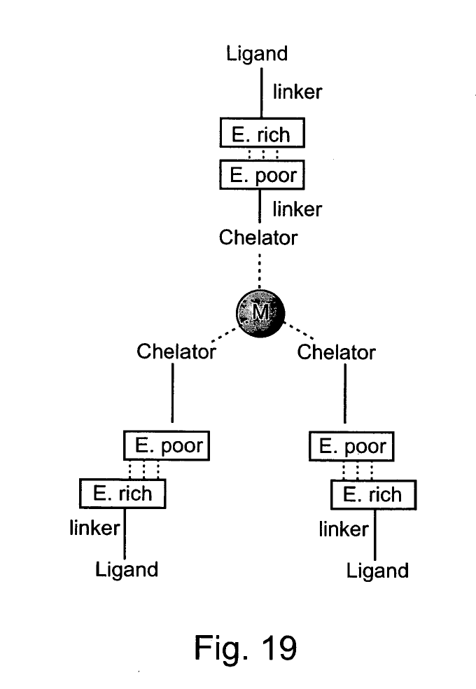

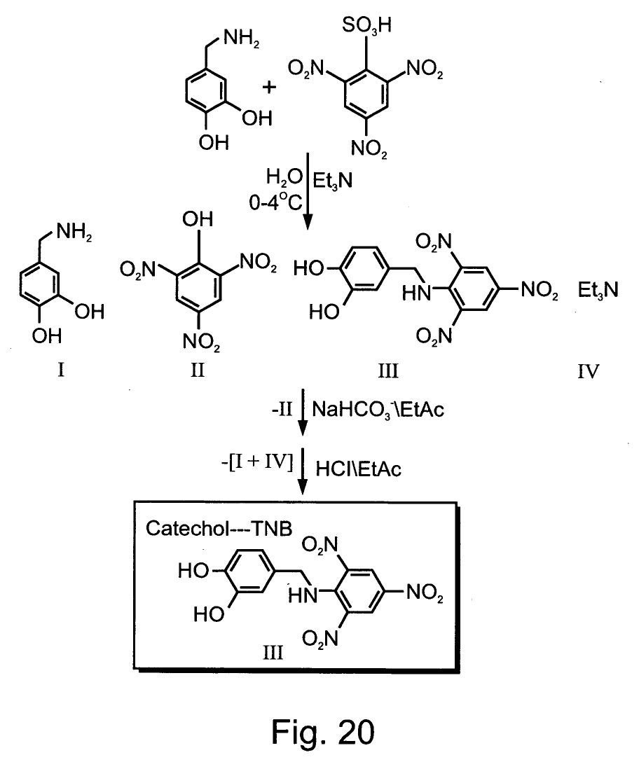

To this end, a chelator that is covalently bound to an electron poor moiety is desired. A synthetic pathway for generating such a combination is presented in FIG. 20.

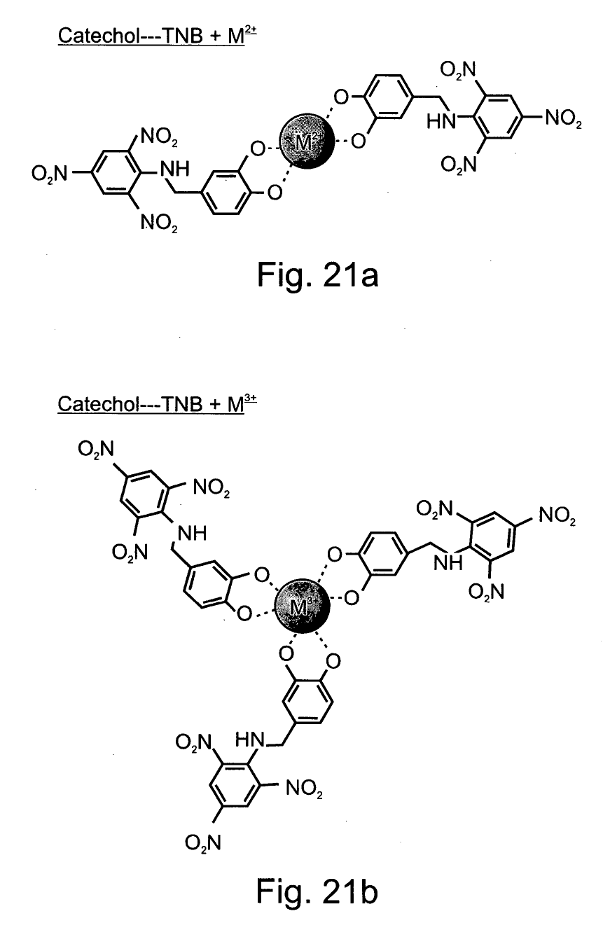

For example, a chelator (e.g. catechol) that is capable to bind both to M²⁺, and M³⁺ metals, is capable in the presence of M²⁺ and M³⁺ metals, to form a non-covalent-di-ligand, (FIG. 21a), or a non-covalent-tri-ligand (FIG. 21b).

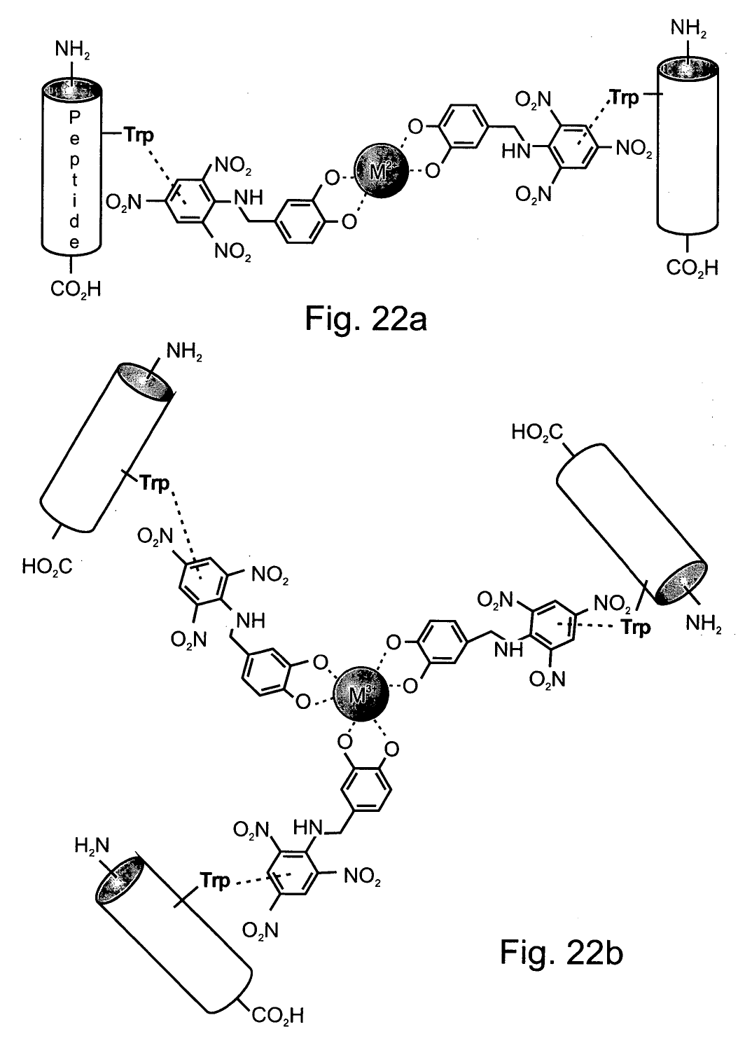

The presence of a peptide (or polypeptide) with a Trp residue (or any other electron rich residue) might lead to the formation of the structures shown in FIGS. 22a-b.

The combination of the two above binding relationships (chelator-metal together with electron rich-poor) may introduce additional advantages. For example, the ability to form non-covalent-multi-ligand-polymeric complexes. This may be achieved by synthesizing two chelators and an electron rich moiety between them (FIG. 23a). In the presence of a ligand----E. poor derivative the complex which is drawn in FIG. 23b is expected to form, which represents a Non-Covalent Polymer of ligands.

Once a dimer, trimer, tetramer etc. is formed, (by a ligand----chelator derivative for example) it may be desired to limit the freedom of motion of the above, in order to achieve more order. If the protein of interest has an electron rich moiety (such as Trp) that is accessible to a covalent di-electron-poor moiety (such as di-trinitrobnezene, TNB---TNB for example) then a complex might be formed between two non-covalent dimers. (FIG. 24). This may lead to the formation of ordered sheets of proteins and multi-ligands.

### Example 4

### The Desthiobiotin-Avidin Platform

Material and Methods

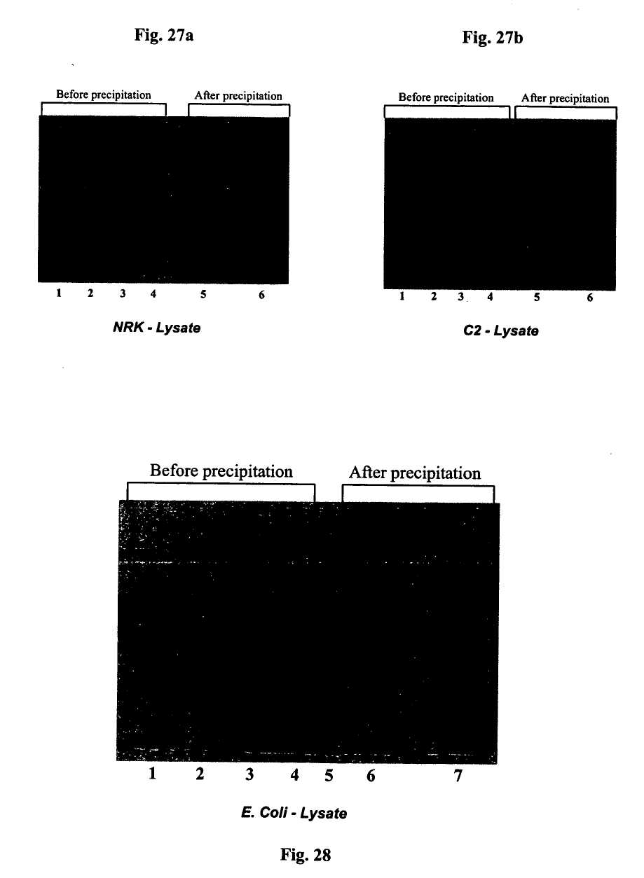

Synthesis of the desthiobiotinylated Protein A (DB-ProA) nonimmobilized ligand. Recombinant Protein A was modified with desthiobiotin N-Hydroxysuccinimidyl ester and yielded the modified Protein A derivative (DB-ProA) utilized in all purification experiments shown in FIGS. 27-29.

Precipitation and elution of rabbit IgG. Precipitation was carried out at 4° C. in a medium containing: 50 mM sodium phosphate at pH 8; 0.23 mg/mL of DB-ProA; 0.6 mg/mL rabbit IgG and cell lysate (either NRK, C2 or E. coli) in a total volume of 50 μL. A freshly prepared avidin solution (1.5 mg/mL final concentration) was added and a precipitate was formed. This was followed by a short spin at 14,000 RPM and removal of the supernatant. The pellet was resuspended once with 200 μL of 50 mM sodium phosphate buffer pH 8 and the supernatant discarded. To elute rabbit IgG, the pellet was further resuspended with 0.1M sodium citrate pH 2.5 or 3, with or without 0.9 M urea at 4° C. for 3-10 minutes in a total volume of 50 μL with or without gentle agitation. After an additional spin, the supernatant was neutralized with 1N NaOH or 3M Tris pH 9 and applied to the gel.

Regeneration of DB-ProA. Recovery of DB-ProA was achieved by incubating the pellets in 0.1M sodium citrate pH 3 and 5 mM of biotin at 4° C. for 10 minutes. Centrifugation at 14,000 RPM was performed and the supernatant was neutralized with 1N NaOH and loaded onto an acrylamide gel.

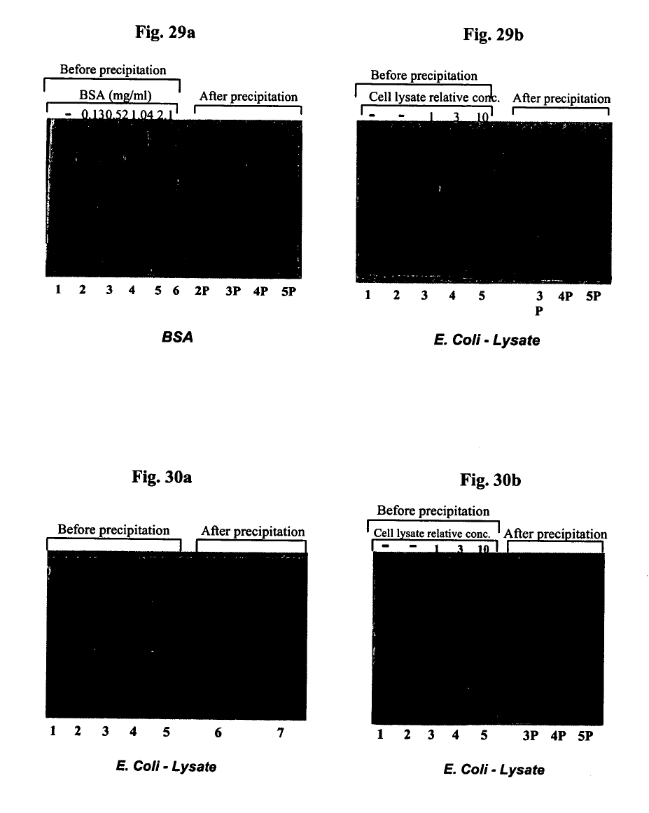

The effect of increased background contamination on the purification process. To study the effect of increased background contamination on the yield and purity of the purification process, identical amounts of rabbit IgG, avidin and DB-ProA were added to increasing concentrations of either BSA (FIG. 29a) or E. coli cell lysate (FIG. 29b). All pellets were washed once with identical volumes of fresh buffer (200 μL) regardless of their contamination background and the IgG was eluted. The eluted IgG solutions exhibited similar purity and yield (FIG. 29a lanes 2P-5P; FIG. 29b, lanes 3P-5P); BSA or E. coli cell lysate served as the background contamination.

Results

Specific precipitation and elution of target proteins. To demonstrate the selectively of the present approach, rabbit IgG was purified from bacterial cell lysates (FIGS. 27-28) by preparing a medium containing whole cell lysate, DB-ProA and rabbit IgG. Upon addition of avidin, a precipitate was generated and the resulting pellet was washed once with 200 μL of fresh buffer. The washed pellet was further incubated under eluting conditions (0.1M sodium citrate at pH 2.5-3, 4° C., for 5 minutes) and the supernatant of the resuspended pellet was applied to the gel after being neutralized to pH 7. The recovery yield of the IgG was 85% (FIG. 27a, lane 5; FIG. 27b, lane 5; FIG. 28, lane 6). Since no DB-ProA was observed by Coomassie staining in the eluted IgG (lane 6), the degree of leached DB-ProA was assessed by silver staining and was determined to be less than 1% (data not shown).

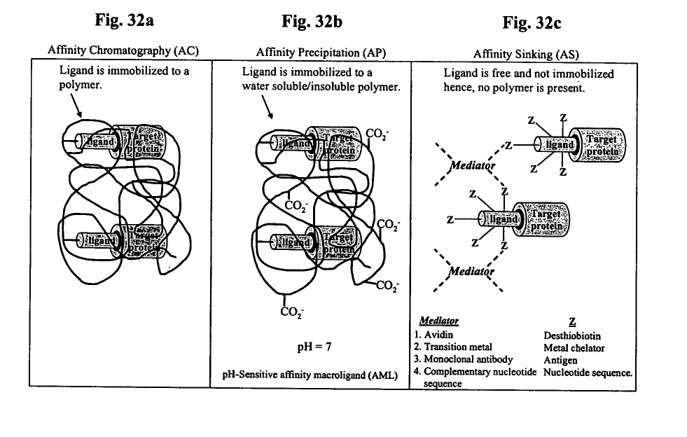

The modified ligands used in this study were desthiobiotinylated protein A (DB-ProA) and desthiobiotinylated concanavalin A (DB-ConA). Incubation of the modified ligand with the target protein and addition of the interconnecting entity (free avidin) generated a precipitate, composed primarily of the [modified ligand-target protein-avidin] multi-complex (FIG. 32c). The target protein is then eluted from the generated percipitate (i.e. pellet) under conditions that essentially do not dissociate the [modified ligand-avidin] multi-complex.

Since antibody purification is a major scientific and industrial need, the present study also tested the ability of the present approach to specifically capture and purify rabbit IgG from different cell lysates, utilizing DB-ProA as the ligand (FIGS. 27-28). The high purity (95-97%) and yield (80-86%) of the recovered IgG, demonstrates the feasibility of the present approach. The majority of impurities are excluded from the pellet in the precipitation step (FIG. 27a, lane 6, FIG. 27b, lane 6; FIG. 28 lane 7) prior to the washing step. This emphasizes the advantage of the present composition, which lacks any polymeric matrix onto which impurities would probably have been adsorbed non-specifically.

Similar precipitation and recovery behavior was observed with a desthiobiotinylated concanavalin A derivative (DB-ConA), used for the capture of glucose oxidase and porcine thyroglobulin (Table 1 below).

TABLE 1
Recovery yields and purity of the target
proteins and the modified ligands

| | Desthiobio- | Recovery | Purity | Recovery yield |
|---|---|---|---|---|
| Target | tinylated | yield of | of target | of desthiobio- |
| protein | ligand | target protein | protein | tinylated ligand |
| Rabbit IgG | Protein A | 80-86% | 97% | 80% |
| Thyro- | Concanavalin A | 70-75% | 95% | 85-89% |
| globulin | | | | |
| Glucose | Concanavalin A | 70-75% | 95% | 85-89% |
| oxidase | | | | |

These consistent results with two distinct ligands indicate that other ligands may be utilized accordingly and lead to highly purified proteins with good recovery yields. Native protein A or concanavalin A lacking bound desthiobiotin did not lead to precipitation of the target proteins (data not shown). The use of non-immobilized ligands may raise the concern of ligand leaching. Nevertheless, leaching was not observed by Coomassie staining (FIG. 27a, lane 5, FIG. 27b lane 5, FIG. 28 lane 6). Therefore gels were visualized by silver staining and the degree of leached DB-ProA was less than 1% (data not shown). Since these values were obtained under eluting conditions at highly acidic conditions (pH 3), one would expect lower levels of leaching under milder eluting conditions. These observations suggest that target proteins can be eluted directly from the generated precipitates, while keeping the [modified ligand-avidin] macro-complex intact in the precipitate. This feature may be advantageous for large-scale protein purification, where obtaining a relatively pure protein in high concentrations by direct elution of the target protein from the pellet is a major advantage (2).

Furthermore, since all ligands utilized by the present approach are modified with a complexing entity (e.g. desthiobiotin, metal chelator) removal of minute amounts (<1%) of leached ligand can be accomplished by passing the sample containing primarily the eluted protein through an appropriate affinity column that would remove traces of leached modified ligand rather than the target protein. For example, a desthiobiotinylated-ligand could be removed from a solution containing the target protein by an avidin column.

Generally, as background contamination increases, greater volumes of buffer are needed to remove impurities that bind non-specifically to the polymeric matrix. Since no polymer matrix is present in the present composition, it is postulated that a major increase in the contamination background would not affect the purity of the eluted protein. Thus, to demonstrate such a phenomenon, all pellets must be washed with minimum and identical volume of buffer, regardless of their background contamination. The results shown in FIG. 29a, lanes 2P-5P; FIG. 29b, lanes 3P-5P, support this speculation and show that a 10 or 16 fold increase in the contamination background has no significant effect on either the purity or the yield of the target protein. Moreover, when pellets were not washed following formation and the IgG was eluted, high purity was obtained, thus providing additional supporting data to the “non-stickiness” nature of the precipitates. These results may imply that other contaminants (e.g. endotoxins, viruses, host DNA) could be excluded by the precipitation step, thereby reducing the number of purification steps in the downstream process.

In the preferred scenario, in which the target protein eluted from the pellet, regeneration of the modified ligand could be accomplished by a simple dialysis procedure. Since desthiobiotin has a lower association constant for biotin binding proteins (Kₐ˜5×10¹³ M⁻¹ for streptavidin) than biotin (Kₐ˜1×10¹⁵ M⁻¹), the pellet will dissociate upon addition of biotin (28). Dialysis will remove excess of unbound biotin, leaving the modified ligand (DB-ProA or DB-ConA) and the [avidin-(biotin)₄] complex in the dialysis container. This mixture (devoid of free biotin) could be used directly in the next batch, since the free [avidin-(biotin)₄] complex is blocked (essentially irreversibly) with 4 biotins, can not participate in network formation, and thus can be considered as an additional contaminant which will be excluded together with all impurities of the next cycle. This procedure was performed for the regeneration of both DB-ProA and DB-ConA (Table 1 above).

The non-immobilized state of the modified ligand might posses additional theoretical advantages which include higher yields of purified product due to faster and more efficient binding to the target protein in homogenous solutions where no additional steric hindrances are imposed by the polymeric matrix. The non-immobilized ligand is expected to be more available for binding, while in its immobilized state may also interact with the polymeric matrix making itself less available for binding. The measured affinity of the modified ligand should represent its affinity upon use, enabling easier judgment as to the most appropriate modified ligand derivative to be utilized in a particular purification process. It has been argued that once a ligand is immobilized its affinity may be reduced by up to a factor of 1000 (30). Such a concern is not relevant to the present approach since no ligand immobilization is required; the amount of added modified ligand to the medium is (theoretically) not limited, whereas affinity columns are characterized by their specific capacity. Therefore, more protein can be purified per batch. Additional benefits deriving from the non-immobilized state may result in higher purity of the end product due to the absence of a polymeric matrix onto which impurities can adsorb; implementation of harsh sanitizing procedures without risking ligand functionality (i.e. the modified ligand can be removed from any instrumentation prior to sanitation); while a dramatic volume reduction within a single precipitation step would enable further purification manipulations with lab-scale machinery.

It will be appreciated that the present approach is fundamentally different from immunoprecipitation. In the latter, antibody-antigen complexes are removed from solution in the presence of an insoluble form of an antibody binding protein such as protein A or an immobilized second antibody, while in the present approach all components (i.e. the modified ligand and the interconnecting entity) are water soluble and are not immobilized.

Essentially, the approach does not introduce a new chemical principle but rather a different chemical architecture which could utilize any ligand, provided that specificity and affinity as well as uniformity are preserved following ligand modification. The possibility of generating equivalent precipitates utilizing other types of modified ligands (e.g. ligand-chelator, ligand-antigen, ligand-nucleotide sequence, (FIG. 32c) emphasizes the wide applicability of the present approach. Furthermore, the [DB-ProA-avidin] complex may serve as a “core complex” for additional applications such as positive/negative cell selection—target cells could be purified (or depleted) with the above “core complex” and an antibody targeted at an epitope on the target cell (FIG. 33a) or depletion of viruses via use of an antibody specific to the virus (FIG. 33b).

### Example 5

### The Metal-Chelator Platform

Materials and Methods

Synthesis of the catechol Protein A derivative (ProA-CAT) nonimmobilized ligand. Recombinant Protein A was modified a N-Hydroxysuccinimidyl ester derivative of the strong metal catechol (catechol-NHS) and yielded the modified Protein A derivative (ProA-CAT) utilized in all purification experiments shown in FIG. 30.

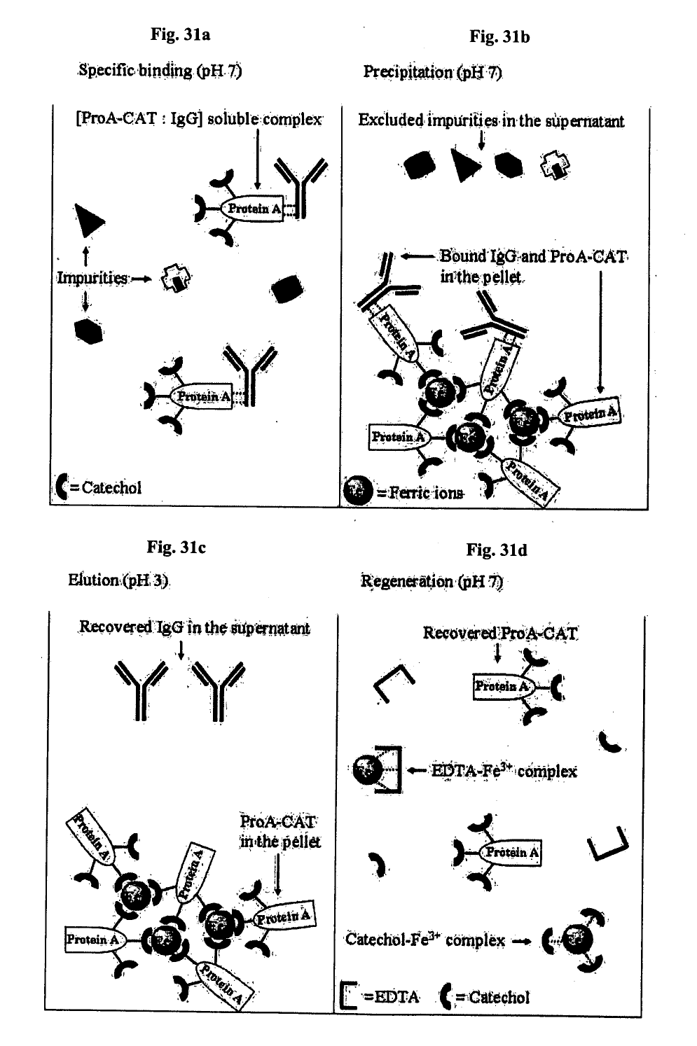

Purification of rabbit IgG from E. coli cell lysate utilizing ProA-CA T and Fe3+ ions (FIGS. 30-31). ProA-CAT (0.46 mg/ml) was added to the E. coli cell lysate (first dialyzed to remove 20 mM imidazole) containing 0.5 mg/ml rabbit IgG, 10 mM NaPi, 400 mM NaCl at pH 7. Following 3-5 minutes of incubation at 4° C., 3 mM of Fe³⁺ ions were added to initiate precipitation of the [ProA-CAT:IgG] soluble complex (FIG. 31b). Two hundred mM of imidazole were added to suppress non-specific interactions between the generated macro-complexes and impurities possessing weak chelating residues (e.g. His, Cys). Following centrifugation at 14,000 RPM, the supernatant primarily contained impurities with no evidence of ProA-CAT and the IgG (FIG. 30a, lane 7). The pellet (containing the complexed IgG) was then washed once with 100 μl of fresh buffer containing 20 mM NaPi pH 7, to remove traces of impurities.

Rabbit IgG was eluted from the washed pellet by resuspending it for 3-5 minutes at 4° C. in 0.4 M Gly and 0.3 M His at pH 3. Following centrifugation at 14,000 RPM, the supernatant was removed and neutralized; analysis thereof revealed presence of the target IgG. The average recovery yield was 80% with a purity greater than 95% as determined via densitometry (FIG. 30a, lane 6). Similar yield and purity results (yield: 71%; purity >95%) were obtained with bovine IgG, thus, demonstrating the applicability of the present approach in purifying targets with lower affinity toward protein A.

The effect of increased background contamination on the purification process. Generally, greater volumes of buffer are required to remove impurities that adsorb non-specifically to polymeric matrixes in chromatographic columns as the contamination increases. Since no polymeric matrixes are utilized by the present approach, it was postulated that an increase in the background contamination should not affect the purity of the recovered IgG. To demonstrate such a phenomenon, constant concentration of rabbit IgG and ProA-CAT were added to increasing concentrations of E. coli cell lysate (FIG. 30b lanes 3-5) and all generated pellets were washed once with a minute volume (100 μl) of buffer regardless of their contamination background. While the recovery yield of the IgG decreased with increased contamination background (˜80% to ˜70-75%), the purity (>95%) was similar (FIG. 30b, lanes 3P-5P), thus emphasizing the advantage of a purification approach lacking a polymeric component.

Regeneration of ProA-CAT. ProA-CAT was regenerated without any chromatographic process at neutral pH in the presence of strong metal chelators such as EDTA and catechol. It was assumed that these chelators will compete with the ProA-CAT on the complexed Fe³⁺ ions, thereby leading to dissolution of the [ProA-CAT:Fe³⁺] macro-complex (FIG. 31d). Indeed, a short incubation at 4° C. in the presence of 50 mM NaPi pH=7, 100 mM EDTA, 50 mM catechol and 10% ethylene glycol lead to quantitative dissolution of the pellet and regeneration of the ProA-CAT in 75-85% yield (data not shown). The free and complexed chelators, together with all other reagents, could then be dialyzed, enabling the reuse of the ProA-CAT.

Thus, a general platform for antibody purification utilizing free nonimmobilized protein A modified with the strong metal chelator catechol (ProA-CAT) and Fe³⁺ ions is presented. The mechanism of purification requires formation and precipitation of macro-complexes composed of: [ProA-CAT:IgG:Fe³⁺]. Target IgGs are eluted from the precipitates at pH 3 in high yields (71-80%) and high purity (>95%), without dissociating the [ProA-CAT:Fe³⁺] insoluble macro-complex.

Highly purified antibody preparations represent a major scientific and industrial need. In a recent study (34) the present inventors presented a novel purification approach, utilizing free nonimmobilized desthiobiotinylated ligands (e.g., protein A; concanavalin A) and free avidin. The nonimmobilized state of the ligand circumvents the need for immobilizing ligands to polymeric supports hence, polymers are excluded from the process and purification is accomplished without chromatographic columns. This study further demonstrated the implementation of the present approach on a novel, more challenging platform, the Metal:Chelator platform. Protein A, a 42 kDa factor produced by several stains of Staphylococcus aureus, which binds specifically to the Fc region of different classes of immunoglobulins (35), was modified with an active ester derivative of the strong metal chelator catechol, catechol-NHS according to Bayer et al. (36). The modified protein A (ProA-CAT) serves as the nonimmobilized ligand and is used for purification of rabbit and bovine IgGs from E. coli cell lysate.

The mechanism of purification of this aspect of the present approach requires three successive steps:

(i) Incubation of the modified ligand (ProA-CAT) with the target IgG to initiate specific binding and formation of the: [ProA-CAT:IgG] soluble complex (FIG. 31a).

(ii) Precipitation of the [ProA-CAT:IgG] complex upon addition of Fe³⁺ ions which generate insoluble macro-complexes composed of: [ProA-CAT:IgG:Fe³⁺], whereas impurities are left in the supernatant and are discarded by centrifugation (FIG. 31b).

(iii) Elution of the IgG from the [ProA-CAT:IgG:Fe 3+] insoluble macro-complex (i.e. pellet) under conditions which essentially do not dissociate the [ProA-CAT:Fe³⁺] macro-complex, thus leading to a simple and fast recovery of the target IgG (FIG. 31c).

Catechol was chosen as the preferred chelator since it: (a) exhibits high affinity toward diverse transition metals (37), therefore enabling the use of a variety of transition metals; (b) requires three independent catechol moieties to chelate a single Fe³⁺ ion, thereby increasing the possibility of interconnecting adjacent [ProA-CAT IgG] soluble complexes; (c) was expected to retain its chelating ability even at acidic conditions (pH 3) due to the absence of basic atoms (e.g. nitrogen) required for complex formation. A nitrogen atom (if existed) would be protonated at low pH and not be available for chelating Fe³⁺ ions.

Several independent results imply that Fe³⁺ ions function as the interconnecting entity: (a) precipitation of the [ProA-CAT:IgG] complex was abolished in the presence of free chelators [e.g., EDTA, catechol, desferal (a specific Fe³⁺ chelator)]; (b) other transition metals (e.g., Cu²⁺, Zn²⁺, Mg²⁺, Ni²⁺) possessing lower affinity toward catechol did not lead to substantial precipitation under identical conditions; and (c) regeneration of ProA-CAT at physiological pH was accomplished only in the presence of strong metal chelators.

In conclusion, the simple precipitation approach presented herein eliminates the need for sophisticated instrumentation (e.g. HPLC) and provides a highly efficient approach for large scale purification of target molecules/cells. In addition, it provides a fast and simple approach and thus would be advantageous in purification of targets that tend to denature rapidly while being highly amenable to scaling by simply increasing the concentration of the modified ligand.

In addition, the present approach enables efficient capture of low abundance targets by simply increasing the modified ligand concentration (being a reagent) without significantly diluting the sample, thereby increasing the rate of complex formation (Rate=k [Free ligand] [Target]). Targets are not diluted within the process (unlike column chromatography) and are eluted into small volumes of elution buffer, resulting in concentrated preparations which may be used directly for crystallization trials. The present approach may be applicable to positive or negative cell selection, virus depletion and immunoprecipitation via epitope capture by a free antibody.

Furthermore, all presently known chromatographic and precipitation techniques require covalent attachment between the ligand and a polymeric support, while the present approach uses ligands in their free non-immobilized state. The use of free ligands circumvents the need for immobilizing ligands to polymers and would exclude polymers from the purification process. FIG. 32 illustrates the differences in chemical architecture between well established approaches (e.g. affinity chromatography, affinity precipitation) and the present approach (labeled as “affinity sinking”), in which, precipitation of the target protein requires two water soluble entities: a modified ligand and an interconnecting entity.

### Example 6

### Synthesis of the Multivalent Nonimmobilized Ovalbumin Ligand

Highly purified ovalbumin (Sigma A5503) was modified with desthiobiotin N-Hydroxysuccinimidyl ester and 6-[Fluorescein-5(6)-carboxamido]hexanoic acid N-hydroxysuccinimide ester (Sigma-F1756) in the following stoichiometric ratio: Ovalbumin:Desthiobiotin:Fluorescein, 1:22:12. Modification was carried out in 0.1M NaHCO3 pH 8.5 for 4 hours at room temperature followed by extensive dialysis to remove excess of free desthiobiotin and fluorescein. The modified ovalbumin serves as the multi-nonimmobilized ligand of the present invention.

Purification of Anti-Fluorescein mAb

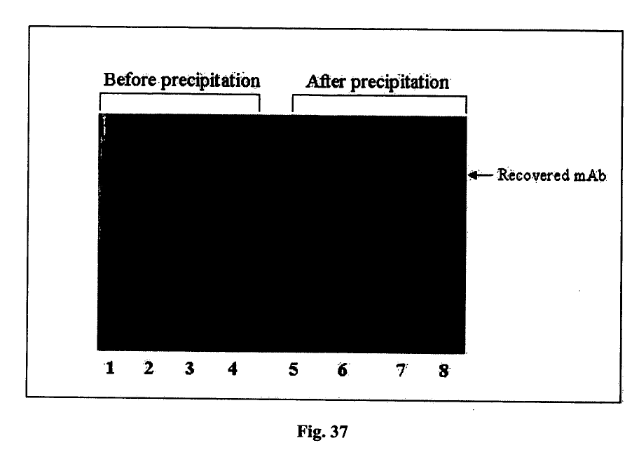

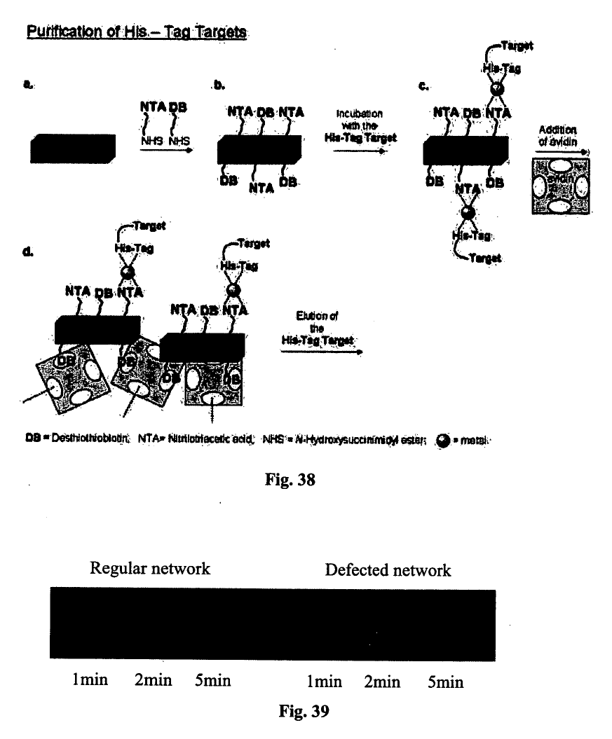

Purification of anti-Fluorescein mAb was carried out at 4° C. in a medium containing: 10-20 mM sodium phosphate at pH 7; 0.5 mg/ml of the modified ovalbumin; 1.2 mg/ml of total protein containing ˜0.1 mg/ml of IgG1 anti-FITC mAb in a total volume of 50 μL. After a short incubation with the modified ovalbumin, a freshly prepared avidin solution (1.5 mg/ml final concentration) was added and a precipitate was formed. This was followed by a short spin at 14K and removal of the supernatant containing the majority of impurities. The content of the supernatant after the addition of avidin is shown in lane 5 of FIG. 37. To demonstrate specific binding between the anti-Fluoresein mAb and the fluorescein immobilized on the ovalbumin, precipitation was performed in the presence of excess free Fluorescein. The presence of the band corresponding to the mAb in lane 6 of FIG. 37 (absent in lane 5) provides direct evidence to a competitive inhibition between of the free and immobilized fluroescein on the target mAb. The pellet was resuspended once with 200 μL of 20 mM sodium phosphate buffer pH 7 and the supernatant containing traces of impurities was discarded. To elute anti-FITC mAb, the pellet was further resuspended with 20 mM sodium phosphate buffer pH 7 and 5 mM of free Fluorescein at 4° C. for 3-10 minutes in a total volume of 50 μL with or without gentle agitation. After an additional spin, the supernatant containing the recovered (i.e. eluted) mAb was neutralized and applied on the gel (lane 7, FIG. 37). Similar recovery the anti-Flourescein mAb was obtained under acidic conditions (0.1M sodium citrate) data not shown. An identical elution procedure was performed on the pellet generated in the presence of free Flourescein. Since no recovered mAb was observed (lane 8, FIG. 37) it imply that most of the mAb was already excluded from the pellet in the precipitation step. The difference in migration between the native (lane 1, FIG. 37) and modified (lane 2, FIG. 37) ovalbumin reflect the degree of modification.

Regeneration of the Modified Ovalbumin

Recovery of modified ovalbumin was achieved by incubating the pellets in 0.1M sodium citrate pH 3 and 5 mM of biotin at 4° C. for 10 minutes. A spin at 14K was performed and the supernatant was neutralized and applied to the gel (data not shown).

### Example 7

### Generation of Modified Ligand Networks

Better eluting efficiency may be accomplished via use of networks/matrices which have “larger holes”. One approach for generation of such networks can be effected by initiating a precipitation process in the presence of free biotin which would occupy some of the binding sites of avidin and avoid maximum interconnections between modified ligands. (e.g. desthiobiotinylated ligand). Similarly, prior incubation of avidin with biotin would be applicable as well.

The upper limit concentration of biotin which does not alter specific precipitation efficiency was identified vby the present inventors and further utilized to evaluate whether faster and more efficient elution is achieved via use of “defective” networks. Porcine thyroglobuline was incubated with desthiobiotinylated concanavalin A (concanavalin A is a known ligand for porcine thyroglobuline) and free D-biotin. After a short incubation free avidin was added and a precipitate was formed thereby forming a defective network. The same procedure was employed in the absence of D-biotin thereby forming a regular, non-defective network. The results suggest faster elution of the target protein (porcine thyroglobuline) from the defective network. (see FIG. 39).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1) Mattiasson, B., Kumar, A., and Galaev, I. Yu. (1998) Affinity precipitation of proteins: design criteria for an efficient polymer. J. Mol. Recognit. 11, 211-216.
2) Freitag, R. (1998) Reversible water-soluble affinity macroligands for bioseparation. Curr. Trends Polymer Sci. 3, 63-79.
3) Freitag, R., and Horvath, C. (1996) Chromatography in the downstream processing of biotechnological products. Adv. Biochem. Bioeng. 53, 17-59.
4) Labrou, N., and Clonis, Y. D. (1994) The affinity technology in downstream processing. J. Biotechnol. 36, 95-119.
5) Lowe, C. R., Lowe, A. R., and Gupta, G. (2001) New developments in affinity chromatography with potential application in the production of biopharmaceuticals. J. Biochem. Biophys. Methods. 49, 561-574.
6) Cuatrecasas, P., Wilchek, M., and Anfinsen, C. B. (1968) Selective enzyme purification by affinity chromatography. Proc. Natl. Acad. Sci. USA 61, 636-643.
7) Cuatrecasas, P., and Wilchek, M. (1968) Single step purification of avidin from egg white by affinity chromatography by biocytin-Sepharose columns. Biochem. Biophys. Res., Commun. 33, 235-239.
8) Clonis, Y. D. (1991) Preparative dye-ligand chromatography. In: M. Hearn (Ed.), HPLC of Proteins Peptides and Polynucleotides, VCH, New York, pp. 453-468.
9) Dyr, J. E., and Suttnar, J. (1997) Separation used for purification of recombinant proteins. J. Chromatogr. B 699, 383-401.
10) Ohlson, S., Hansson, L., Larsson, P-O., and Mosbach, K. (1978) High performance liquid affinity chromatography (HPLAC) and its application to the separation of enzymes and antigens. FEBS Lett. 93, 5-9.
11) Mattiasson, B., and Olson, U, (1986) General chromatographic procedure based on the use of heterobifunctional affinity ligands. J Chromatogr. 370, 21-28.
12) Afeyan, N., Gordonni, N., Mazsaroff, I., Varady, L., Fulton, S., Yang, Y. B., and Regnier, F. (1990) Flow through particles for the high performance liquid chromatographic separation of biomolecules: perfusion chromatography. J. Chromatogr. 519, 1-29.
13) Teichberg, V. I. (1990) Affinity repulsion chromatography. J. Chromatogr. 510, 49-57.
14) Slinerland, R., and Scouten, W. (1990) Centrifugal affinity chromatography. J. Chromatogr. 510, 205-211.
15) Nilsson, J., Ståhl, S., Lundeberg, J., Uhlén, M., and Nygren, P. Å. (1997) Affinity Fusion Strategies for Detection, Purification, and Immobilization of Recombinant Proteins. Protein Expression Purif. 11, 1-16.
16) Schneider, M., Guillot, C., and Lamy, B. (1981) The affinity precipitation technique. Ann. NY Acad. Sci 369, 257-263.
17) Pearson, J., Clonis, Y. D., and Lowe, C. R. (1989) Preparative affinity precipitation of 1-lactate dehydrogenase. J. Biotechnol. 11, 267-274.
18) Gupta, M. N., and Mattiasson, B. (1994). Affinity precipitation in Highly Selective Separations in Biotechnology, ed. By G. Street, pp. 7-33. Chapman and Hall, London.
19) Hilbrig, F., and Freitag, R. (2003) Protein purification by affinity precipitation. J. Chromatogr. B 790, 79-90.
20) Galaev, I. Yu., Gupta, M. N., and Mattiasson, B. (1996) Use smart polymers for Bioseparation CHEMTEC 19-26.
21) Galaev, I. Y. and Mattiasson, B. (1993) Affinity thermoprecipitation: the critical role of polymer in ligand-protein interactions. 6^{th} European Congress on Biotechnology, Florence, 13-17 Jun., 1993, Italy, Vol. 1. M0099.
22) Vaidya, A. A., Lele, B. S., Kulkarni, M. G., and Mashelkar, R. A. (1999) Enhancing ligand-protein binding affinity thermoprecipitation elucidation of spacer effects. Biothechnol. Bioeng. 64, 418-425.
23) Garret-Flaudy F. and Freitag, R. (2001) Use of the avidin (imino) biotinsystem as a general approach to affinity precipitation. Biotechnol. Bioeng. 71, 223-234.
24) Graille, M., Stura, E. A., Corper, A. L., Sutton, B. J., Taussig, M. J., Charbonnier, J. B., Silverman, G. J., (2000) Crystal structure of a Staphylococcus aureus protein A domain complexed with the Fab fragment of a human IgM antibody: structural basis for recognition of B-cell receptors and superantigen activity. Proc. Natl. Acad. Sci. USA 97, 5399-404.
25) Sharon, N., and Lis, H. (1972) Cell-Agglutinating and Sugar Specific Proteins. Science 177, 949-959.
26) Goldstein, I., Hollerman, C., and Smith E. (1965) Protein-Carbohydrate Interaction. II Inhibition Studies on the Interaction of Concanavalin A with Polysaccharides. Biochemistry. 4, 876-883.
27) Grimaldi, J., and Sykes, B. (1975) Concanavalin A: A Stopped Flow Nuclear Magnetic Resonance Study of Conformational Changes Induced by Mn²⁺, Ca²⁺, and alpha-Methyl-D-Mannoside. J. Biol. Chem. 250, 1618-1624.
28) MÜller, W., Ringsdorf, H., Rump, E., Wildburg, G., Zhang, X., Angermaier, L., Knoll, W., Liley, M., and Spinke, J. (1993) Attempts to mimic docking processes of the immune system: recognition-induced formation of protein multilayers. Science 262, 1706-1708.
29) Hirsch, J. D., Eslamizar, L., Filanoski, B. J., Malekzadeh, N., Haugland, R. P., Beechem, J. M., and Haugland, R. P. (2002) Easily reversible desthiobiotinbinding to streptavidin, avidin, and other biotin-binding proteins: uses for protein labeling detection and isolation. Anal. Biochem. 308 (2), 343-357.
30) Kumar, A., and Gupta, M. N. (1994) Affinity precipitation of trypsin with soybean trypsin inhibitor linked Eudragit S-100. J. Biotechnol. 37, 185-189.
31) Bradford, M. M., (1976) A rapid sensitive method for the quantitation of micrograms quantities of protein utilizing the principle of protein-dye binding. Anal. Biochem. 72, 248-254.
32) Laemmli, U. K. (1970) Cleavage of structural proteins during the assembly of the head of bacteriophage T4. Nature 227, 680-685.
33) Bayer, E. D., Wilchek, M., and Skutelsky, E. (1976) Affinity cytochemistry: the localization of lectin and antibody receptors on erythrocytes via the avidin-biotin complex. FEBS Lett. 68, 240-244.
34) Patchornik, G., & Albeck, A., Bioconjugate Chem. 13, 111-119 (2005).
35) Nilsson, J. et al., Protein Expr. Purif. 11, 1-16 (1997).
36) Bayer, E. D., Wilchek, M. & Skutelsky, E., FEBS Lett. 68, 240-244 (1976).
37) Martell, A. E., & Smith, R. M., in Critical Stability Constants, 3, 303-304, Plenum Press, New York (1974).
38) Martell, A. E. & Smith, R. M. in Critical Stability Constants, 3, 200-202, Plenum Press, New York (1974).

What is claimed is:

1. A composition of matter comprising at least one ligand capable of binding a target molecule or cell of interest, said at least one ligand being attached to at least one coordinating moiety selected capable of directing the composition of matter to form a non-covalent complex when co-incubated with a coordinator ion or molecule.

2. The composition of matter of claim 1, wherein said at least one ligand is designed such that said target molecule or cell is bound by predetermined number of ligand molecules of said at least one ligand.

3. The composition of matter of claim 1, wherein said at least one ligand is designed such that said target molecule or cell is bound by a single ligand molecule of said at least one ligand.

4. The composition of claim 1, wherein said complex is a polymeric complex.

5. The composition of claim 1, further comprising said coordinator ion or molecule.

6. The composition of claim 1, wherein said target molecule of interest is selected from the group consisting of a protein, a nucleic acid sequence, a small molecule chemical and an ion.

7. The composition of claim 1, wherein said target cell of interest is selected from the group consisting of a eukaryotic cell, a prokaryotic cell and a viral cell.

8. The composition of claim 1, wherein said at least one ligand is selected from the group consisting of a growth factor, a hormone, a nucleic acid sequence, an antibody, an epitope tag, an avidin, a biotin, a enzymatic substrate and an enzyme.

9. The composition of claim 1, wherein said at least one ligand is attached to said at least one coordinating moiety via a linker.

10. The composition of claim 1, wherein said coordinating moiety is selected from the group consisting of a biotin, a nucleic acid sequence, an epitope tag, an electron poor molecule and an electron-rich molecule.

11. The composition of claim 1, wherein said coordinating moiety is a chelator.

12. The composition of claim 1, wherein said coordinator ion or molecule is selected from the group consisting of an avidin, a nucleic acid sequence, an electron poor molecule and an electron-rich molecule.

13. The composition of claim 1, wherein said coordinator ion or molecule is a metal ion.

14. A method of purifying a target molecule or cell of interest, the method comprising:
(a) contacting a sample including the target molecule or cell of interest with a composition including:
(i) at least one ligand capable of binding the target molecule or cell of interest, said at least one ligand being attached to at least one coordinating moiety; and
(ii) a coordinator capable of non-covalently binding said at least one coordinating moiety, said at least one coordinating moiety and said coordinator being capable of forming a complex when co-incubated; and
(b) collecting a precipitate including said complex bound to the target molecule or cell of interest, thereby purifying the target molecule or cell of interest.

15. The method of claim 14, wherein the molecule of interest is selected from the group consisting of a protein, a nucleic acid sequence, a small molecule chemical and an ion.

16. The method of claim 14, wherein the target cell of interest is selected from the group consisting of a eukaryotic cell, a prokaryotic cell and a viral cell.

17. The method of claim 14, wherein said at least one ligand is selected from the group consisting of a growth factor, a hormone, a nucleic acid sequence, an antibody, an epitope tag, an avidin, a biotin, a enzymatic substrate and an enzyme.

18. The method of claim 14, wherein said at least one ligand is attached to said at least one coordinating moiety via a linker.

19. The method of claim 14, wherein said coordinating moiety is selected from the group consisting of a chelator, a biotin, a nucleic acid sequence, an epitope tag, an electron poor molecule and an electron-rich molecule.

20. The method of claim 14, wherein said coordinator ion or molecule is selected from the group consisting of a metal ion, an avidin, a nucleic acid sequence, an electron poor molecule and an electron-rich molecule.

21. The method of claim 14, further comprising recovering the molecule of interest from said precipitate.

22. A method of detecting predisposition to, or presence of a disease associated with a molecule of interest in a subject, the method comprising contacting a biological sample obtained from the subject with a composition including:
(i) at least one ligand capable of binding the molecule of interest, said at least one ligand being attached to at least one coordinating moiety; and
(ii) a coordinator capable of non-covalently binding said at least one coordinating moiety, said at least one coordinating moiety and said coordinator being capable of forming a complex when co-incubated,
wherein formation of said complex including the molecule of interest is indicative of predisposition to, or presence of the disease associated with the molecule of interest in the subject.

23. The method of claim 22, wherein the molecule of interest is selected from the group consisting of a protein, a nucleic acid sequence, a small molecule chemical and an ion.

24. The method of claim 22, wherein said at least one ligand is selected from the group consisting of a growth factor, a hormone, a nucleic acid sequence, an antibody, an epitope tag, an avidin, a biotin, a enzymatic substrate and an enzyme.

25. The method of claim 22, wherein said at least one ligand is attached to said at least one coordinating moiety via a linker.

26. The method of claim 22, wherein said coordinating moiety is selected from the group consisting of a chelator, a biotin, a nucleic acid sequence, an epitope tag, an electron poor molecule and an electron-rich molecule.

27. The method of claim 22, wherein said coordinator ion or molecule is selected from the group consisting of a metal ion, an avidin, a nucleic acid sequence, an electron poor molecule and an electron-rich molecule.

28. A composition for crystallizing a molecule of interest, the composition comprising:
(i) at least one ligand capable of binding the molecule of interest, said at least one ligand being attached to at least one coordinating moiety; and
(ii) a coordinator capable of non-covalently binding said at least one coordinating moiety, wherein said at least one coordinating moiety and said coordinator are capable of forming a complex when co-incubated and whereas the composition is selected so as to define the relative spatial positioning and orientation of the molecule of interest when bound thereto, thereby facilitating formation of a crystal therefrom under inducing crystallization conditions.

29. The composition of claim 28, wherein the molecule of interest is selected from the group consisting of a protein, a nucleic acid sequence, a small molecule chemical and an ion.

30. The composition of claim 28, wherein said at least one ligand is selected from the group consisting of a growth factor, a hormone, a nucleic acid sequence, an antibody, an epitope tag, an avidin, a biotin, a enzymatic substrate and an enzyme.

31. The composition of claim 28, wherein said at least one ligand is attached to said at least one coordinating moiety via a linker.

32. The composition of claim 28, wherein said coordinating moiety is selected from the group consisting of a chelator, a biotin, a nucleic acid sequence, an epitope tag, an electron poor molecule and an electron-rich molecule.

33. The composition of claim 28, wherein said coordinator ion or molecule is selected from the group consisting of a metal ion, an avidin, a nucleic acid sequence, an electron poor molecule and an electron-rich molecule.

34. A method of crystallizing a molecule of interest, the method comprising contacting a sample including the molecule of interest with a crystallizing composition including:
(i) at least one ligand capable of binding the molecule of interest, said at least one ligand being attached to at least one coordinating moiety; and
(ii) a coordinator capable of non-covalently binding said at least one coordinating moiety, wherein said at least one coordinating moiety and said coordinator are capable of forming a complex when co-incubated and whereas said crystallizing composition is selected so as to define the relative spatial positioning and orientation of the molecule of interest when bound thereto, thereby facilitating formation of a crystal therefrom under inducing crystallization conditions.

35. The method of claim 34, wherein the molecule of interest is selected from the group consisting of a protein, a nucleic acid sequence and a small molecule chemical.

36. The method of claim 34, wherein said at least one ligand is selected from the group consisting of a growth factor, a hormone, a nucleic acid sequence, an antibody, an epitope tag, an avidin, a biotin, a enzymatic substrate and an enzyme.

37. The method of claim 34, wherein said at least one ligand is attached to said at least one coordinating moiety via a linker.

38. The method of claim 34, wherein said coordinating moiety is selected from the group consisting of a chelator, a biotin, a nucleic acid sequence, an epitope tag, an electron poor molecule and an electron-rich molecule.

39. The method of claim 34, wherein said coordinator ion or molecule is selected from the group consisting of a metal, an avidin, a nucleic acid sequence, an electron poor molecule and an electron-rich molecule.

40. A composition-of-matter comprising a molecule having a first region capable of binding a molecule of interest and a second region capable of binding a coordinator ion or molecule, said second region being designed such that said molecule forms a polymer when exposed to said coordinator ion or molecule.

41. The composition of claim 40, wherein said second region is capable of binding more than two coordinator ions or molecules.

42. The composition of claim 40, wherein binding of said coordinator ion or molecule is non covalent binding.

43. The composition of claim 40, wherein said coordinator ion is a metal ion.

44. A method of depleting a target molecule or cell of interest from a sample, the method comprising:
(a) contacting the sample including the target molecule or cell of interest with a composition including:
(i) at least one ligand capable of binding the molecule of interest, said at least one ligand being attached to at least one coordinating moiety; and
(ii) a coordinator capable of non-covalently binding said at least one coordinating moiety, said at least one coordinating moiety and said coordinator being capable of forming a complex when co-incubated; and
(b) removing a precipitate including said complex bound to the target molecule or cell of interest to thereby deplete the target molecule or cell of interest from the sample.

45. The method of claim 44, wherein the molecule of interest is selected from the group consisting of a protein, a nucleic acid sequence, a small molecule chemical and an ion.

46. The method of claim 44, wherein the target cell of interest is selected from the group consisting of a eukaryotic cell, a prokaryotic cell and a viral cell.

47. The method of claim 44, wherein said at least one ligand is selected from the group consisting of a growth factor, a hormone, a nucleic acid sequence, an antibody, an epitope tag, an avidin, a biotin, a enzymatic substrate and an enzyme.

48. The method of claim 44, wherein said at least one ligand is attached to said at least one coordinating moiety via a linker.

49. The method of claim 44, wherein said coordinating moiety is selected from the group consisting of a chelator, a biotin, a nucleic acid sequence, an epitope tag, an electron poor molecule and an electron-rich molecule.

50. The method of claim 44, wherein said coordinator ion or molecule is selected from the group consisting of a metal ion, an avidin, a nucleic acid sequence, an electron poor molecule and an electron-rich molecule.

51. A method of enhancing immunogenicity of a target molecule of interest, the method comprising contacting the target molecule of interest with a composition including:
(i) at least one ligand capable of binding the target molecule of interest, said at least one ligand being attached to at least one coordinating moiety; and
(ii) a coordinator capable of non-covalently binding said at least one coordinating moiety,
wherein contacting is effected such that said at least one coordinating moiety and said coordinator forms a complex including the target molecule of interest, thereby enhancing immunogenicity of the target molecule of interest.

52. The method of claim 51, wherein the molecule of interest is selected from the group consisting of a protein, a nucleic acid sequence, a small molecule chemical and an ion.

53. The method of claim 51, wherein said at least one ligand is selected from the group consisting of a growth factor, a hormone, a nucleic acid sequence, an antibody, an epitope tag, an avidin, a biotin, a enzymatic substrate and an enzyme.

54. The method of claim 51, wherein said at least one ligand is attached to said at least one coordinating moiety via a linker.

55. The method of claim 51, wherein said coordinating moiety is selected from the group consisting of a chelator, a biotin, a nucleic acid sequence, an epitope tag, an electron poor molecule and an electron-rich molecule.

56. The method of claim 51, wherein said coordinator ion or molecule is selected from the group consisting of a metal ion, an avidin, a nucleic acid sequence, an electron poor molecule and an electron-rich molecule.
